(19) United States
(12) Patent Application Publication
(10) Pub. No.: US 2026/0209681 A1
(43) Pub. Date: Jul. 23, 2026

(54) ARTICLES, SYSTEMS, AND METHODS FOR TRAPPING AND/OR LYSING CELLS AND OTHER ENTITIES

(71) Applicants: President and Fellows of Harvard College, Cambridge, MA (US); BLASTID, Inc., Boston, MA (US)

(72) Inventors: Johan Paulsson, Cambridge, MA (US); Youlian Plamenov Goulev, Cambridge, MA (US); Thomas Feltrup, Boston, MA (US); Paulo A. Garcia, Boston, MA (US)

(73) Assignees: President and Fellows of Harvard College, Cambridge, MA (US); BLASTID, Inc., Boston, MA (US)

(21) Appl. No.: 19/425,683

(22) Filed: Dec. 18, 2025

Related U.S. Application Data
(60) Provisional application No. 63/737,557, filed on Dec. 20, 2024.

(51) Int. Cl. C12M 1/00; C12M 3/06

(52) U.S. Cl. CPC C12M 47/06; C12M 23/16; C12M 47/04

(57) ABSTRACT

The present disclosure generally relates to various articles, systems, and methods for trapping cells, e.g., within a microfluidic device. Some aspects describe microfluidic channels that prevent certain entities such as microbial cells from flowing through the channels, while allowing certain cells (e.g., blood cells) to pass. In one set of embodiments, for example, a sample (e.g., a blood sample) having a plurality of mammalian cells and a plurality of microbial cells is flowed through the microfluidic devices described herein, resulting in retention of the microbial cells.

### RELATED APPLICATIONS

This application claims the benefit of U.S. Provisional Patent Application Ser. No. 63/737,557, filed Dec. 20, 2024, by Paulsson, et al., incorporated herein by reference. The government has certain rights in this invention.

### GOVERNMENT FUNDING

This invention was made with government support under 1AY2AX000005-01 awarded by U.S. Department of Defense/Defense Advanced Research Projects Agency (DOD/DARPA).

### TECHNICAL FIELD

Articles, systems, and methods for trapping cells and/or lysing cells are generally described.

### BACKGROUND

Microbial cells, such as bacteria, viruses, and fungi, can be present at abnormal levels in fluids, but detecting them may have certain challenges. For example, some diagnostic techniques for detecting bacteria in blood are often time consuming, and may be ineffective at isolating bacteria. In addition, conventional diagnostic techniques for isolating microbial cells may require pre-processing steps such as centrifugation, which may further restrict the ability to process samples efficiently. Thus, improvements are needed.

### SUMMARY

The subject matter of the present disclosure involves, in some cases, interrelated products, alternative solutions to a particular problem, and/or a plurality of different uses of one or more systems and/or articles.

Certain embodiments are directed to a device (e.g., microfluidic device) for separating one or more biological components [e.g., one or more infectious agents (e.g., one or more viruses, one or more bacteria, one or more fungi, one or more prokaryotic cell, one or more eukaryotic cell, or a combination thereof)] [e.g., one or more target cells (e.g., one or more non-blood cell, one or more mammalian cell, one or more human cell, one or more animal cell, one or more patient cell, one or more circulating tumor cell, one or more sickle cell, one or more rare cell, or a combination thereof)] in a fluid, the device comprising a plurality of biological component separation units fluidically connected in parallel, the units comprising channels (e.g., micro-and/or nano-channels) arranged (e.g., sized, shaped, and/or oriented) such that fluid flow through the units causes separation of biological components having different sizes and/or mechanical properties [e.g., by lysing one biological component and not another biological component (e.g., by trapping said another biological component)] [e.g., by trapping one biological component (e.g., at an entrance to a channel in the unit) and allowing another biological component to pass through and/or be lysed].

Certain embodiments are directed to a device (e.g., microfluidic device) for lysing one or more biological components [e.g., cells (e.g., blood cells and/or epithelial cells)] in a fluid, the device comprising a plurality of biological component lysing units fluidically connected in parallel, the units comprising channels (e.g., micro-and/or nano-channels) arranged (e.g., sized, shaped, and/or oriented) such that fluid flow through the units causes lysing of one or more biological components (e.g., one or more target cells).

Certain embodiments are directed to a device (e.g., microfluidic device) for separating and/or lysing one or more biological components in a fluid, the device comprising first channels (e.g., microchannels) (e.g., fluidically connected in parallel to each other) and second channels (e.g., micro-and/or nano-channels) (e.g., a subset of which is fluidically connected in parallel to each first channel) arranged such that fluid flow through the device travels through the first channels then the second channels, wherein the first channels are larger than the second channels.

Certain embodiments are directed to a method comprising, in a microfluidic device, mechanically lysing cells using micro and/or nano-channels (e.g., first channels) thereby releasing virus, binding the virus to particles (e.g., in a reservoir of a device disposed in a fluid pathway after a first set of micro-and/or nano-channels), and trapping the particles with the micro-and/or nano-channels.

Certain embodiments are directed to a device comprising first biological component separation units connected in parallel, wherein the biological component separation units are arranged (e.g., sized, shaped, and/or oriented) to trap a first biological component in a fluid; and second biological component separation units connected in parallel, wherein the first biological component separation units are fluidically connected in series with the second biological component separation units.

Certain embodiments are directed to a method of separating (e.g., isolating) one or more biological components [e.g., one or more infectious agents (e.g., one or more viruses, one or more bacteria, one or more fungi, one or more prokaryotic cells, one or more eukaryotic cells, or a combination thereof)] (e.g., cells) in a fluid (e.g., blood, urine, lymphatic fluid, saliva, sputum) and/or purifying a fluid, the method comprising flowing a fluid comprising a first biological component through channels (e.g., microchannels) in a device (e.g., a microfluidic device) such that at least 50% of the first biological component in the fluid that flows through the channels is trapped in and/or at the channels (e.g., one or more entrances thereto).

Certain embodiments are directed to a method of separating (e.g., isolating) one or more biological components [e.g., one or more infectious agents (e.g., one or more viruses, one or more bacteria, one or more fungi, or a combination thereof)] in a fluid (e.g., blood, urine, lymph, saliva, sputum) lysing a biological component in a fluid, and/or purifying a fluid, the method comprising flowing a fluid comprising a cellular biological component (e.g., through a device); and applying (e.g., via suction) one or more pressures (e.g., one or more pressure gradients) to the biological component with the channels while the fluid is flowing, thereby lysing the biological component.

Certain embodiments are directed to a method of (e.g., selectively) lysing cells (e.g., blood cells), the method comprising mechanically lysing cells in a fluid by flowing the fluid through channels in a device.

Certain embodiments are directed to an article, comprising a microfluidic device defining a plurality of repeat units, each of the repeat units comprising a first microfluidic channel, and at least 3 second channels (e.g. nanofluidic channels) each fluidically connecting to the first microfluidic channel, wherein the first microfluidic channel has a maximum cross-sectional dimension less than 5 micrometers, and wherein the second channels have a maximum cross-sectional dimension less than 0.5 micrometers.

Certain embodiments are directed to an article, comprising a microfluidic device defining a plurality of repeat units, each of the repeat units comprising a first microfluidic channel, a collection microfluidic channel substantially parallel to the first microfluidic channel, and at least 3 substantially parallel second channels (e.g., nanochannels) each fluidically connecting the first microfluidic channel to the collection microfluidic channel, wherein the first microfluidic channel has a maximum cross-sectional dimension less than 5 micrometers, and wherein the second channels have a maximum cross-sectional dimension less than 0.5 micrometers.

Certain embodiments are directed to a method, comprising providing a fluid containing a first type of cells and second entities, wherein the first type of cells has an average volume bigger than the second entities; distorting the first type of cells to have an aspect ratio of at least 21; and flowing the fluid through a microfluidic channel such that the first type of cells pass therethrough and the second entities do not pass therethrough.

Certain embodiments are directed to a method, comprising providing a fluid containing a first type of cells and second entities, wherein the first type of cells has an average volume bigger than the second entities; distorting the first type of cells to have an aspect ratio of at least 21; and applying a force to the distorted first type of cells to cause the first type of cells to pass through a microfluidic channel dimensioned to prevent the second entities from flowing therethrough.

Certain embodiments are directed to a method, comprising providing a fluid containing a first type of cells and a second entities, wherein the first type of cells has an average volume bigger than the second entities; flowing the fluid through a first microfluidic channel having a volume less than 120% of the first type of cells and a maximum cross-sectional dimension less than 60% of the average diameter of a perfect sphere having the same volume as the first type of cells; flowing the first type of cells through a second microfluidic channel intersecting with the first microfluidic channel without flowing the second entities through the second microfluidic channel.

Certain embodiments are directed to a method, comprising flowing a fluid containing a first type of cells and a second entities through a microfluidic channel having a cross-sectional diameter smaller than a diameter of a perfect sphere having the same volume as the type of cells having the smaller volume, such that the first type of cells pass therethrough and the second entities do not pass therethrough.

Certain embodiments are directed to a method, comprising flowing a fluid containing blood cells and bacteria through a microfluidic filter such that at least 90% of the bacteria are trapped by the microfluidic filter and at least 90% of the blood cells pass through the microfluidic filter; and backflushing a second fluid through the microfluidic filter to recover at least 90% of the bacteria trapped by the microfluidic filter.

Certain embodiments are directed to a method, comprising providing a fluid containing a first type of cells and second entities, wherein the first type of cells has an average volume bigger than the second entities; and flowing the fluid through a microfluidic filter such that at least 90% of the second entities are trapped by the microfluidic filter and at least 90% of the first type of cells pass through the microfluidic filter.

Certain embodiments are directed to a method, comprising providing a fluid containing a first type of cells and second entities, wherein the first type of cells has an average volume bigger than the second entities; and flowing the fluid through a microfluidic filter such that at least 90% of the second entities are trapped by the microfluidic filter and at least 90% of the first type of cells are lysed by the microfluidic filter.

Certain embodiments are directed to a method, comprising providing a fluid containing cells and particles, wherein cells have an average volume bigger than the particles; and flowing the fluid through a microfluidic filter such that at least 90% of the particles are trapped by the microfluidic filter and at least 90% of the cells pass through the microfluidic filter.

Certain embodiments are directed to a method, comprising providing a fluid containing cells and particles, wherein cells have an average volume bigger than the particles; and flowing the fluid through a microfluidic filter such that at least 90% of the particles are trapped by the microfluidic filter and at least 90% of the cells are lysed by the microfluidic filter.

Certain embodiments are directed to a method of determining cells; comprising passing a fluid comprising a first type of cells suspected of containing therein a second type of cells through a microfluidic filter such that the first type of cells are lysed and the second type of cells are trapped by the microfluidic filter; and determining a distribution of the second type of cells within the microfluidic filter to determine containment of the second type of cells within the first type of cells.

Certain embodiments are directed to a method of determining a viral infection; comprising passing a fluid comprising cells suspected of being infected by a virus through a microfluidic filter such that the cells are lysed and the viruses are trapped by the microfluidic filter; and determining a distribution of the viruses within the microfluidic filter to determine infection of the cells by the virus.

Certain embodiments are directed to a method, comprising distorting a mammalian cell in a first direction such that it has an aspect ratio of at least 21; distorting the mammalian cell in a second direction different from the first direction; and flowing the distorted mammalian cell through a microfluidic channel.

Certain embodiments are directed to a method, comprising distorting a mammalian cell in a first direction such that it has an aspect ratio of at least 21; and applying a force to the mammalian cell in a second direction different from the first direction sufficient to move the mammalian cell in the second direction.

Certain embodiments are directed to a method of lysing a cell, comprising distorting a mammalian cell such that it has an aspect ratio of at least 21; and applying a force to the distorted mammalian cell sufficient to lyse the cell.

Certain embodiments are directed to a method, comprising confining a mammalian cell inside of a first microfluidic channel having a volume less than 120% of the cell and a maximum cross-sectional dimension less than 60% of the average diameter of a perfect sphere having the same volume as the cell; and flowing the confined mammalian cell through a second microfluidic channel intersecting with the first microfluidic channel.

Certain embodiments are directed to a method, comprising confining a mammalian cell inside of a first microfluidic channel having a volume less than 120% of the cell and a maximum cross-sectional dimension less than 60% of the average diameter of a perfect sphere having the same volume as the cell; and applying a force to a stretched portion of the confined mammalian cell by flowing a fluid through a second microfluidic channel.

Certain embodiments are directed to a method of lysing a cell, comprising confining a mammalian cell inside of a first microfluidic channel having a volume less than 120% of the cell and a maximum cross-sectional dimension less than 60% of the average diameter of a perfect sphere having the same volume as the cell; and applying a force to the confined mammalian cell sufficient to lyse the cell.

Certain embodiments are directed to a method, comprising confining a mammalian cell inside of a first microfluidic channel such that it has an aspect ratio of at least 21; and flowing the confined mammalian cell through a second microfluidic channel intersecting with the first microfluidic channel.

Certain embodiments are directed to a method, comprising confining a mammalian cell inside of a first microfluidic channel such that it has an aspect ratio of at least 21; and applying a force to a stretched portion of the confined mammalian cell by flowing a fluid through a second microfluidic channel.

Certain embodiments are directed to a method of lysing a cell, comprising confining a mammalian cell inside of a first microfluidic channel such that it has an aspect ratio of at least 21; and applying a force to the confined mammalian cell sufficient to lyse the cell.

Certain embodiments are directed to a method of (e.g., selectively) lysing cells (e.g., blood cells), the method comprising distorting (e.g., deforming) a cell by flowing fluid comprising the cell dispersed therein; and applying pressure to the cell while the cell is distorted thereby lysing the cell. In some cases, the device is a microfluidic device.

Other advantages and novel features of the present disclosure will become apparent from the following detailed description of various non-limiting embodiments of the disclosure when considered in conjunction with the accompanying figures. In cases where the present specification and a document incorporated by reference include conflicting and/or inconsistent disclosure, the present specification shall control.

### BRIEF DESCRIPTION OF THE DRAWINGS

Non-limiting embodiments of the present disclosure will be described by way of example with reference to the accompanying figures, which are schematic and are not intended to be drawn to scale unless otherwise indicated. In the figures, each identical or nearly identical component illustrated is typically represented by a single numeral. For purposes of clarity, not every component is labeled in every figure, nor is every component of each embodiment of the disclosure shown where illustration is not necessary to allow those of ordinary skill in the art to understand the disclosure. In the figures:

FIG. 1A illustrates a cell distorted in a first microfluidic channel, according to one set of embodiments.

FIG. 1B illustrates a cell distorted in a second microfluidic channel, according to another set of embodiments.

FIG. 1C illustrates a first type of cell and a second entity flowing through microfluidic channels, according to yet another set of embodiments.

FIG. 1D illustrates a microfluidic design, according to certain embodiments.

FIGS. 2-5 illustrate various aspects of a microfluidic device, according to some embodiments.

FIGS. 6 and 7 illustrate more than one repeating unit within a microfluidic device, according to certain embodiments.

FIG. 8 illustrates microfluidic and/or nanofluidic channels within a microfluidic device, according to certain embodiments.

FIG. 9 illustrates an example of a microfluidic device with an inlet and an outlet, according to certain embodiments.

FIG. 10 illustrates Malassez counting chambers, according to certain embodiments.

FIG. 11 illustrates a mean number of colony-forming units (CFUs) on the plates was compared with the mean number of bacteria trapped and detected in the microfluidic device, according to certain embodiments.

FIG. 12 illustrates the ability to reverse the direction of the flowing cells through a microfluidic device, according to some embodiments.

FIG. 13 illustrates cells passing through certain microfluidic channels, according to certain embodiments.

FIGS. 14 and 15 illustrate blood spiked with E. coli in a microfluidic device, according to certain embodiments.

FIGS. 16-18 illustrate microfluidic chips retaining different bacteria and related cell plates, according to certain embodiments.

FIG. 19 illustrates isolated bacteria from a urine sample, according to certain embodiments.

FIG. 20 illustrates Escherichia coli isolated within trench geometries in a filter-type chip architecture and sequentially imaged using brightfield, hyperspectral imaging, and quantitative phase microcopy, according to certain embodiments. This also demonstrates that once a microfluidic channel is filled it can continue filtering and accumulating components within the main feeding channel.

FIG. 21 illustrates bacterial cell retention rate of a microfluidic filter tested with E. coli, according to certain embodiments.

FIG. 22 illustrates flow reversal to unload cells trapped in a microfluidic device, according to certain embodiments

FIG. 23 illustrates trapping a fluorescently labeled E. coli cells, according to some embodiments.

FIG. 24 illustrates repeating units in a microfluidic device, according to some embodiments.

FIGS. 25A-25D illustrate the trapping and multi-modal imaging of Candida albicans, in another embodiment.

FIG. 26. illustrates staphylococcus aureus that is physically isolated within trench geometries in a filter-type chip architecture and sequentially imaged using (A) brightfield, (B) hyperspectral imaging (HSI), and (C) quantitative phase microscopy (QPM). Images were captured in the same field of view sequentially (FOV) on an integrated imaging system, according to some embodiments.

FIG. 27A-27H illustrates cell viability in certain buffers, according to certain embodiments.

FIGS. 28A-B illustrate a microfluidic device trapping Escherichia coli bacteria, according to illustrative embodiments of the present disclosure.

### DETAILED DESCRIPTION

The present disclosure generally relates to various articles, systems, and methods for trapping cells, e.g., within a microfluidic device. Some aspects describe microfluidic channels that prevent certain entities such as microbial cells from flowing through the channels, while allowing certain cells (e.g., blood cells) to pass. In one set of embodiments, for example, a sample (e.g., a blood sample) having a plurality of mammalian cells and a plurality of microbial cells is flowed through the microfluidic devices described herein, resulting in retention of the microbial cells.

When flowing a fluid through the microfluidic channels, certain entities (e.g., second entities) of the fluid may get trapped within the microfluidic channels, which allow a separation of these entities from other components (e.g., a first type of cells) in the fluid. These second entities may be a second type of cells, such as microbial cells, for example, and the first type of cells may be mammalian cells, such as blood cells. In some cases, the first type of cells are larger than the entities (e.g., the second type of cells). In some cases, the second entities may be further analyzed. For example, in embodiments where the second entities comprise bacterial cells, it may be possible to determine the bacterial cell identity (e.g., a bacterial strain), among other cell properties. Examples of such analytical techniques include, but are not limited to, those described in U.S. Pat. Apl. Ser. Nos. 63/646,582, 63/646,599, and 63/646,534, each incorporated herein by reference in its entirety.

In certain cases, the present disclosure teaches microfluidic channels that are configured to distort (e.g., squeeze, stretch, and/or bend) certain cells (e.g., mammalian cells), e.g., that are contained in a fluid, whereas other cells in the fluid (e.g., bacterial cells) may not get so distorted. In one aspect, cells are flowed into a first microfluidic channel, where the cells may get confined. In some cases, the confined cells may flow to a second microfluidic channel, and in some cases, this may further distort the cells. These cells may experience mechanical stress, which may cause the cells to be distorted. In some cases, such distorted cells may be able to pass through the microfluidic channels. In some cases, the cells may be lysed, although in certain embodiments, such distorted cells may pass through intact.

Certain aspects of the present disclosure describe microfluidic channels such as a first microfluidic channel and a second microfluidic channel in certain arrangements or having certain dimensions. Some embodiments are generally directed to microfluidic devices containing repeat units that may contain copies of such microfluidic channels, e.g., to permit larger volumes of cells to be processed. In some cases, the repeat units may comprise a first microfluidic channel and a second microfluidic channel, e.g., as described herein. In some embodiments, some or all of the microfluidic channels of the repeat units within the microfluidic device may not be substantially clogged (e.g., with cell debris), for example, such that fluid can no longer pass through the microfluidic device or such that fluid flow rate is impracticably low (e.g., such that a device can no longer serve its intended purpose), after flowing a fluid.

Certain aspects of the present disclosure are directed to a microfluidic device configured to separate certain cells (e.g., microbial cells) or entities (e.g., second entities) present in a fluid, e.g., containing a first type of cells. In one aspect, a plurality of cells comprises a first type of cells and a second entity (e.g., a second type of cells), where the second entities may get stuck or trapped when flowed through a microfluidic device, e.g., as discussed herein. In another aspect, a fluid flows from a first microfluidic channel to a second microfluidic channel, such that some second entities get stuck or trapped before flowing through the second microfluidic channel. For example, the second entities may comprise microbial cells, fungal cells, viruses, particles, or a combination thereof that may get stuck or trapped before flowing through certain microfluidic channels (e.g., first or second microfluidic channels). Other examples of second entities are discussed in more detail herein.

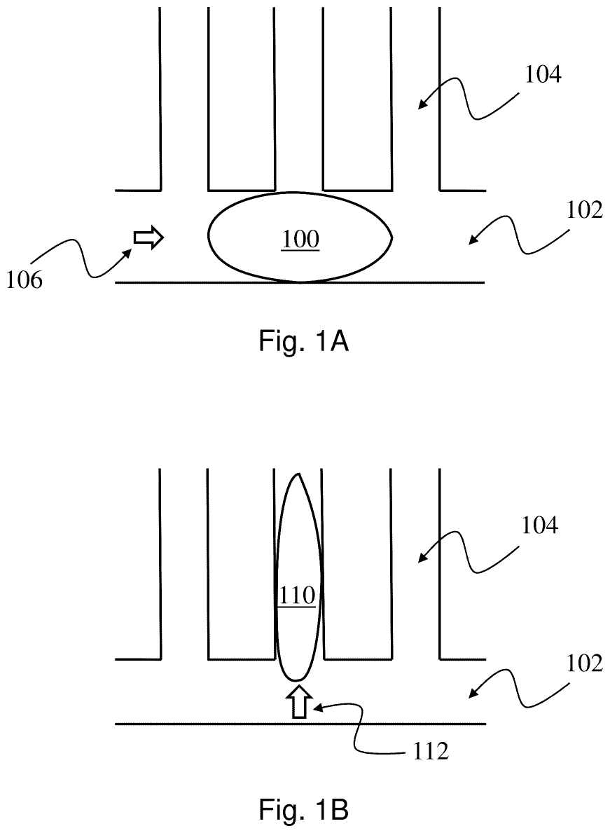

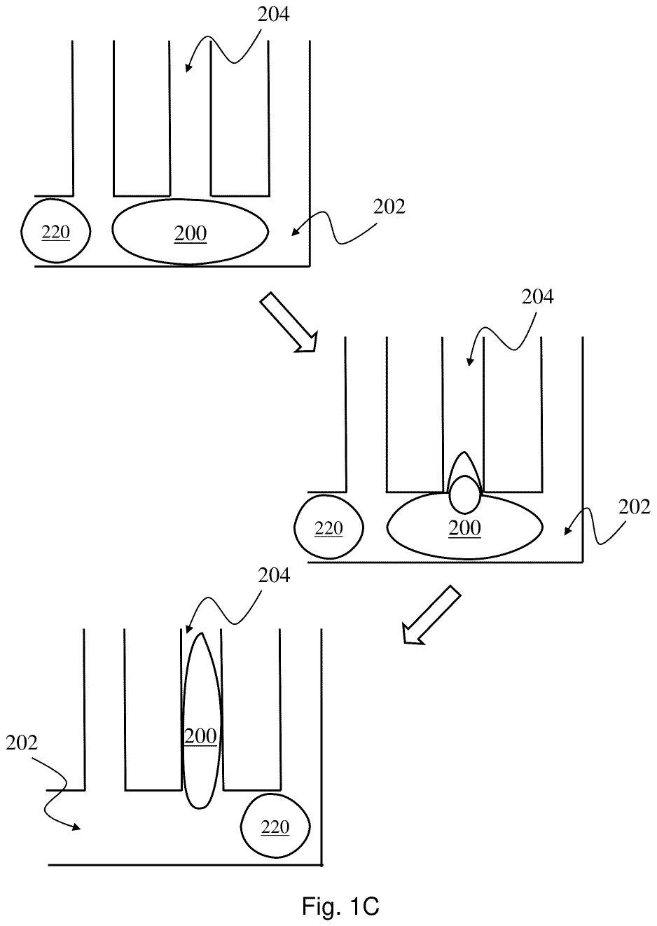

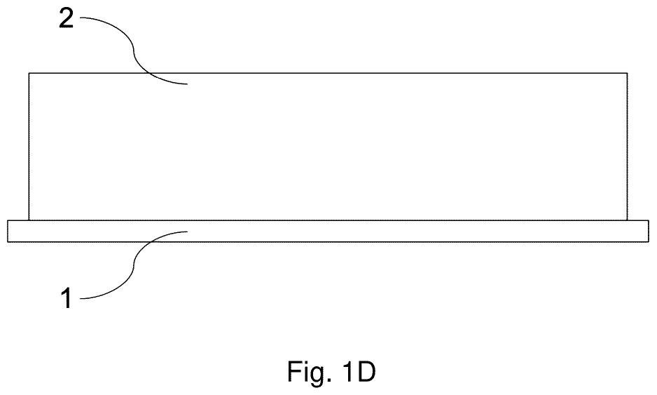

As an example, FIG. 1C illustrates cell 200 of a first type of cells and cell 220 of a second type of cells in first microfluidic channel 202, where cell 200 of a first type of cells may flow to second fluidic channel 204, whereas cell 220 of a second type of cells may get stuck in first fluidic channel 202. Although 220 is described as a cell of a second type of cell, in other embodiments, 220 may be another second entity, e.g., as discussed herein.

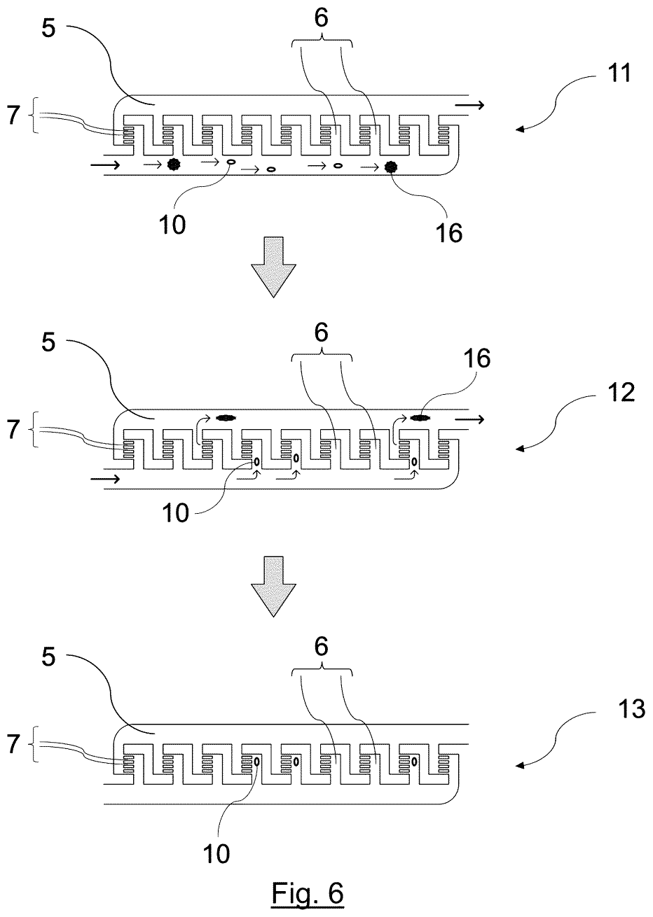

In addition, as another non-limiting example, as seen in FIG. 6, cell 16 of a first type of cells and cell 10 of a second type of cells (or another second entity, such as discussed herein) may flow through a microfluidic channel within a microfluidic device, where cell 10 of the second type of cells (or other second entity) may get trapped while cell 16 of the first type of cells may pass therethrough. In certain cases, the second type of cells or other second entities may not flow through some channels (e.g., second microfluidic channel) of a microfluidic size due to an inability to be distorted, for example. In some cases, the second type of cells or other second entities may have certain mechanical properties (e.g., high rigidity) that may prevent the cell from flowing through certain channels, as described later herein.

Another aspect of the present disclosure describes trapping certain entities, such as cells within the microfluidic device. In one aspect, certain microbial cells or other second entities may be trapped in the microfluidic device after flowing a fluid (e.g., a plurality of cells). Some microbial cells (e.g., bacteria, viruses, or fungi) may not be lysed inside the microfluidic device. In some embodiments, a plurality of cells having microbial cells and mammalian cells are flowed through a microfluidic device, such that the mammalian cells pass through and/or are lysed while the microbial cells (e.g., and other entities) do not. In some embodiments, a plurality of cells having first cells [e.g., first mammalian cells (e.g., circulating tumor cells)] and second cells [e.g., second mammalian cells (e.g., blood cells)] are flowed through a microfluidic device, such that the second cells pass through and/or are lysed while the first cells do not. In some embodiments, the mammalian cells are lysed (which may facilitate their passage through the microfluidic device), although in some embodiments, the mammalian cells are not lysed. In certain cases, the diameter of the microfluidic channel may be smaller than the diameter of the mammalian cells and/or the diameter of the microbial cells or other entities.

In one aspect, smaller cells or other entities (e.g., bacterial cells) may get trapped, while larger cells (e.g., mammalian cells) are not trapped. As a non-limiting example, a bacterial cell such as E. Coli may have a diameter that is larger than a cross-sectional diameter of a second microfluidic channel (e.g., at least 2 micrometers) and may not flow through such channel, whereas a mammalian cell such as a human red blood cell may have a diameter larger than the cross-sectional diameter of the channel but may be able to flow through such channel. In such example, the human red blood cell may be able to be distorted (e.g., stretched) in a microfluidic channel whereas the E. coli may not. The human red blood cell may or may not be lysed, but is able to pass through the microfluidic channel (e.g., either intact or in the form of lysate components) while the E. Coli is not, which is surprising when considering that the human red blood cell is actually larger than the E. Coli cell.

Certain microfluidic devices described herein may have a suitable trapping efficiency for a second type of cells, such as microbial cell, or other second entities. The trapped cells may be present after flowing a fluid comprising such cells. For example, in some embodiments, the microfluidic device may trap at least 50%, at least 60%, at least 70%, at least 80%, at least 90%, at least 95%, at least 98%, at least 99%, or at least 99.5% of the second entities after flowing a fluid. In some cases, no more than 10% (e.g., no more than 5%, no more than 3%, no more than 2%, no more than 1%, no more than 0.5%, no more than 0.1%, no more than 0.01%, or no more than 0.001%) of a second biological component, or other second entity in a fluid that flows through the device is trapped (e.g., clogged) in the device.

In some aspects, a cell such as a mammalian cell is distorted in a microfluidic device. Although the above discussion described certain configurations involving first and second microfluidic channels (e.g., microchannels and/or nanochannels), which are discussed in more detail herein, it should be understood that the present disclosure is not so limited, and other embodiments, more generally, are directed to various articles, systems, and methods for distorting cells, which may aid in distinguishing, separating, and/or trapping, first type of cells from a second type of cells, first type of cells from a second entities.

In certain embodiments, distorting (e.g., squeezing, deforming, compression, torsion, and/or bending) the cells may occur when the cells are subject to mechanical stress caused by the conformation, shape, or dimensions of some of the channels or other regions within a microfluidic device. For example, the stresses may be caused by mechanical elements within the microfluidic device (e.g., channels, ports, inlets, outlets, chambers, or substrates), by fluid forces within the microfluidic device (e.g., fluid flows acting upon cells, by external forces applied to the microfluidic device (e.g., pressure forces, gravitational forces, or centrifugal forces).

Some embodiments are generally directed to mammalian cells distorted in a first direction. The first direction may be any suitable direction, for example, in a direction defined by a microfluidic channel within the microfluidic device. For example, as seen in FIG. 1A, mammalian cell 100 may be distorted with respect to the microfluidic device in a direction (e.g., first direction 106) defined by the microfluidic channel (e.g., first microfluidic channel 102). In addition, according to certain embodiments, the cell may be distorted in a second direction. The second direction may be different from the first direction. As seen in FIG. 1B, mammalian cell 110 may be distorted in second direction 112 defined by second microfluidic channel 104. In some cases, the first direction is perpendicular or orthogonal to the second direction, however, other configurations or angles are possible. The cell may be further distorted in a second direction after being distorted in the first direction, according to certain embodiments. In some cases, the force applied to the cell as a result of the microfluidic channel configuration and/or dimensions may be higher or lower in the second direction than the first direction. In some cases, the force applied to the cell in the second direction may be sufficient to lyse the cells, although this is not necessary.

According to one set of embodiments, the mammalian cells in a first direction may be confined in a confining microfluidic channel. Mammalian cells that are so confined may in certain cases, be subsequently distorted in a second direction, for example, by fluid flow in a second direction. In some aspects, the first direction is defined by a first microfluidic channel, and the second direction is defined by a second microfluidic channel.

Some of the mammalian cells may be distorted (e.g., stretched) inside the microfluidic device when being flowed. According to some embodiments, the stretching of the cells may occur such that the cells exhibits an aspect ratio of at least 2:1, at least 3:1, at least 4:1, or at least 5:1. In some embodiments, the aspect ratio of the cells may be no more than 5:1, no more than 4:1, no more than 3:1, or no more than 2:1. Combinations of these ranges are possible.

The cells may be confined within some microfluidic channels of the microfluidic device, in accordance with certain embodiments. For example, according to certain embodiments, mammalian cells inside a first microfluidic channel are confined to a certain volume and/or a certain dimension. In one set of embodiments, a microfluidic channel (e.g., first microfluidic channel) may have a volume at least 500%, at least 400%, at least 300%, at least 250%, at least 200%, at least 150%, at least 140%, at least 130%, at least 120%, at least 110%, or at least 100% of the cell. In another set of embodiments, the maximum cross-sectional dimension (e.g., cross-sectional diameter) of the microfluidic channel (e.g., first microfluidic channel) may have a maximum cross-sectional dimension no more than 80%, no more than 70%, no more than 60%, no more than 50%, or no more than 40% of the average diameter of a perfect sphere having the same volume as the cell. Combinations of these ranges are possible. The first microfluidic channel may be a confining microfluidic channel.

The cells being confined within a microfluidic channel (e.g., first microfluidic channel) may be distorted before being flowed into another microfluidic channel (e.g., second microfluidic channel. In some cases, it may be advantageous to have distorted cells (e.g., mammalian cells) being flowed into a second microfluidic channel (e.g., for further distortion). In certain embodiments, a force (e.g., a second force) is applied to a stretched portion of the confined mammalian cell when being flowed through a second microfluidic channel. The force being applied to the second portion may be sufficient to lyse some cells (e.g., mammalian cells). The force being applied to a cell in the second microfluidic channel may be higher than a force being applied to the cell (e.g., confined cell) in the first microfluidic channel, according to some embodiments.

Certain cells may be trapped after being flowed through the microfluidic device. In some embodiments, cells that are not lysed (e.g., bacteria) are trapped, while cells that are lysed (e.g., human blood cells) may be fractured and thus may release cell components that are smaller than the diameter of the cell, which may flow better through the microfluidic channels. Lysing some cells in the microfluidic device may allow cell components to flow out (e.g., flow out through an outlet) of the microfluidic device. It is also possible that, according to some embodiments, mammalian cells are able to be squeezed within some microfluidic channels of the microfluidic device and therefore may flow through device without getting trapped.

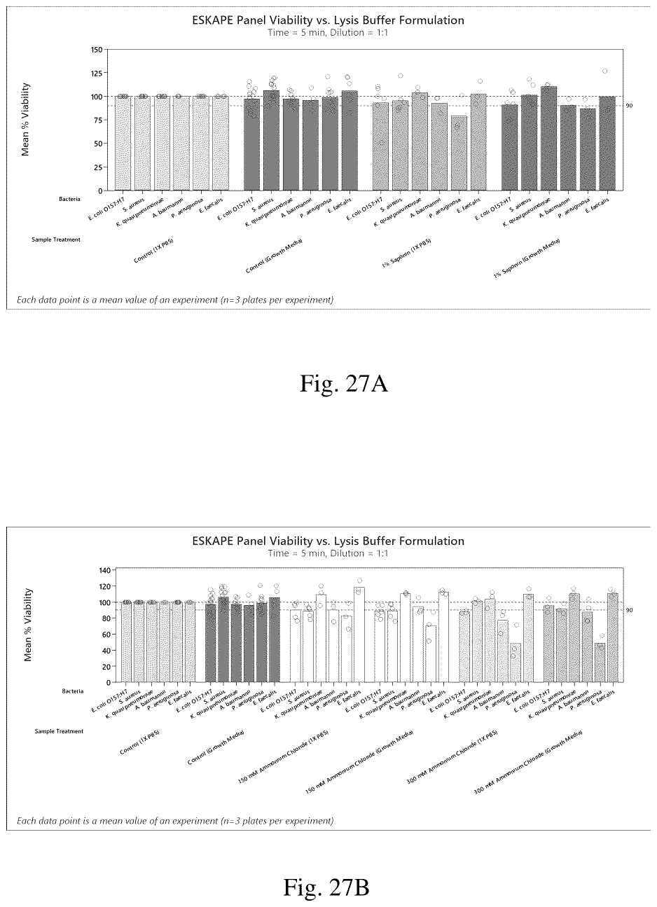

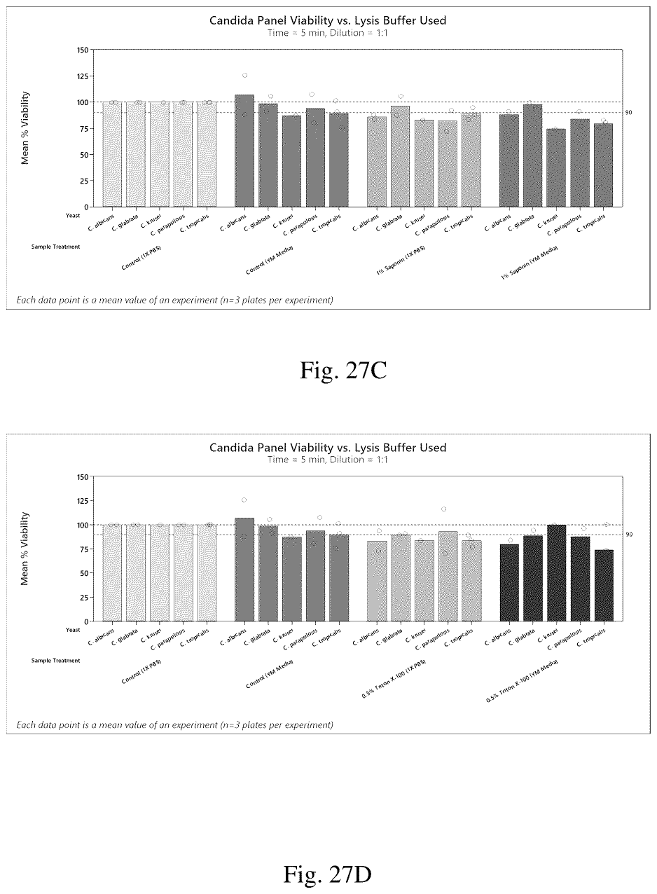

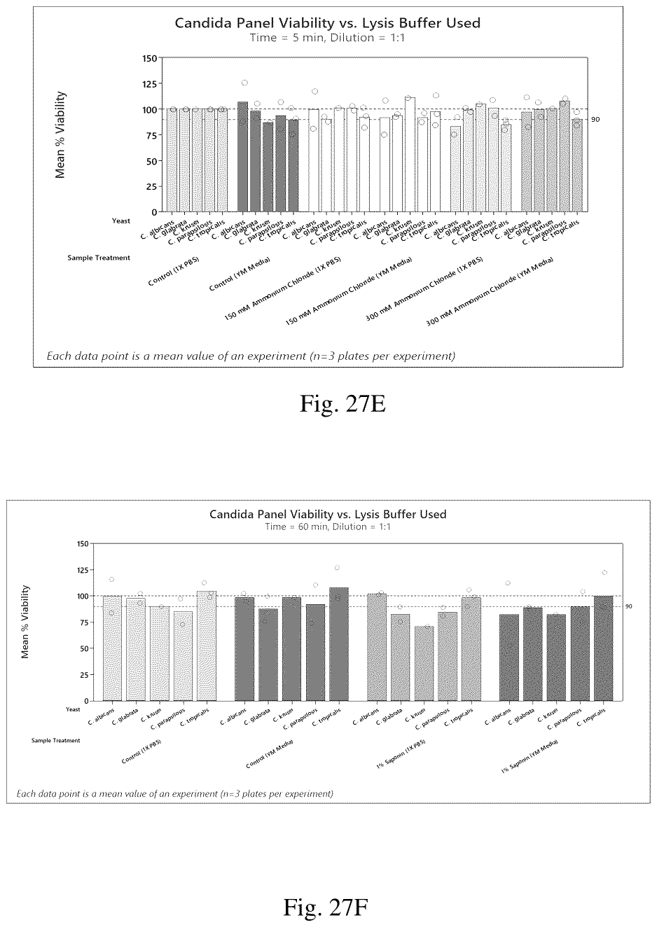

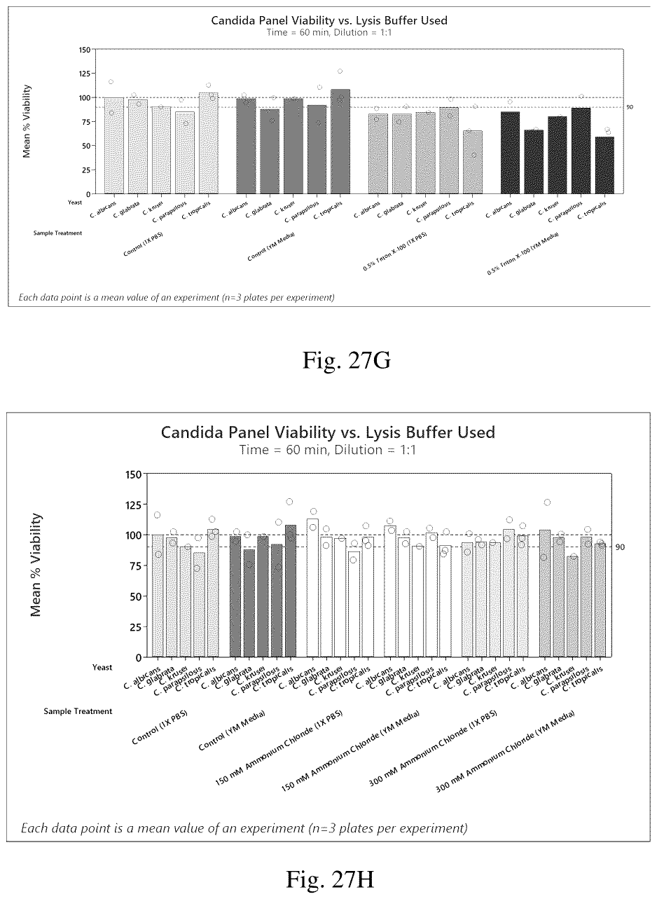

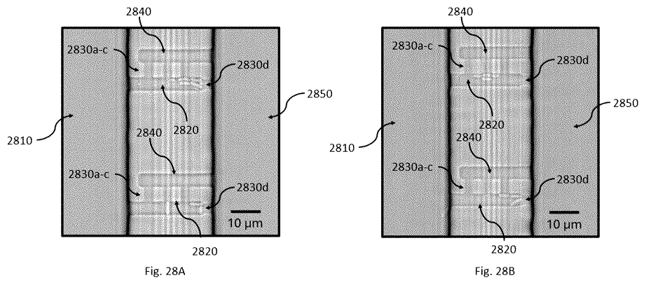

In certain embodiments, a plurality of cells having cells that can be lysed and cells that cannot be lysed (e.g., by a microfluidic device) are flowed through a microfluidic device. Non-limiting example data regarding viability of certain cells in lysis buffers is shown in FIG. 27A-27H. The cells may be flowed through the microfluidic device and be exposed to lysing conditions, which may cause a portion of the cells (e.g., mammalian cells) to be lysed. An example of lysing conditions could be flowing the cells through some of the microfluidic devices described herein. Another example is exposing the cells to a lysing composition. After lysing some cells, the portion of the cells that are not lysed can be collected (e.g., removed through an opening of the microfluidic device). In some embodiments, the cells collected are smaller than the cells lysed. The cells trapped within the microfluidic device may be collected, analyzed, or further processed. Some examples of techniques used to analyze or process the cells are described later herein.

Distorting the cell (e.g., mammalian cell) may cause cell lysing in some cases. In certain cases, a force is applied to the cell (e.g., to a side of the cell) that is at least sufficient to lyse the cell. The force may be generated by the microfluidic channel configuration, shape, such that the cell is mechanically distorted. Although a force applied may cause the cells to lyse, other methods may be used in other embodiments to cause lysing, in addition to and/or instead of applying a force to the cell. For example, in some cases, friction between the cell (e.g., mammalian cell) and the microfluidic channel walls may be used to lyse the cells. In some cases, a lysing composition may be used to cause cell lysing, e.g., as described later herein in more detail.

According to some aspects, a device may include one or more repeat units or separation units, e.g., biological component separation units. The repeat units may comprise, for example, a first microfluidic channel and a second microfluidic channel. In one set of embodiments, there may be a plurality of substantially parallel second microfluidic channels that are fluidically connected to the first channel.

In some cases, the first channels have a width and/or a height in a range of from 500 nm to 15 μm (e.g., from 1 μm to 10 μm). In addition, in certain embodiments, the first channels may have a length of at least 10 μm (e.g., at least 20 μm, at least 30 μm, at least 40 μm, or at least 50 μm). In some cases, the first channels can have a size corresponding to a biological component (e.g., one or more infectious agents (e.g., one or more viruses, one or more bacteria, one or more fungi, one or more prokaryotic cell, one or more eukaryotic cell, or a combination thereof)), or other second entities.

In some cases, the second channels have a width and/or a height in a range of from 100 nm to 500 nm. In certain embodiments, the second channels have a length of at least 500 nm (e.g., at least 1 μm, at least 2 μm, at least 3 μm, or at least 4 μm). In some cases, the second microfluidic channels may be relatively small, e.g., having a cross-sectional dimension of less than 1 micrometer. In some cases, the second channels have a size smaller than a biological component or a second entity.

The second channels may be fluidically connected to one or more third or collection microchannels or microfluidic channels (e.g., wherein the repeat units comprise the third microchannels). In some cases, the third channels may be larger than the second channels. In certain embodiments, fluid flow through the device travels through the second channels then the third microchannels. The third channels may, in some cases, be disposed parallel to the first channels. In addition, in some cases, a collection fluidic channel is connected to the first microfluidic channel through one or more (e.g., at least 3) of the substantially parallel microfluidic channels, e.g., mutually parallel. The collection fluidic channel may be substantially parallel to the first microfluidic channel, for example. In some cases, some or all of the first microfluidic channels may have at least one dimension (e.g., a cross-sectional dimension, e.g., relative to a direction of fluid flow) that is larger than a corresponding at least one dimension of some or all of the second microfluidic channels. In some cases, the third channels may have the same or different size as the first channels. In some cases, a ratio of the at least one dimension to the corresponding at least one dimension is in a range of from 1 to 1000 (e.g., is at least 1.1, at least 1.2, at least 1.25, at least 1.3, at least 1.4, at least 1.5, at least 1.6, at least 1.7, at least 1.75, at least 1.8, at least 1.9, at least 2, at least 2.5, at least 3, at least 4, at least 5, at least 6, at least 7, at least 8, at least 9, at least 10, at least 20, at least 30, at least 40, at least 50, at least 60, at least 70, at least 80, at least 90, at least 100, at least 200, at least 300, at least 400, at least 500, at least 600, at least 70, at least 800, at least 900, or at least 1000).

As discussed herein, the third channels may be directly fluidically coupled to a common fluid collection channel, e.g., connected to an outlet. In addition, in some cases, there may be a common fluid distribution channel, e.g., where the first channels are directly fluidically coupled to the common fluid distribution channel, for instance, connected to an inlet. In some cases, the first channels and the second channels are arranged such that all flow from the inlet to the outlet travels through the first channels and then the second channels.

In one set of embodiments, the device may include a first microfluidic channel and a collection channel, with a plurality of second channels fluidically connecting them. The second channels may be parallel (e.g., mutually parallel) or non-parallel. In some embodiments, the parallel channels fluidically connecting the first microfluidic channel and the collection channel may be at least 1, at least 2, at least 3, at least 5, at least 10, at least 20, at least 50, at least 100, or at least 500. In some embodiments, the parallel channels fluidically connecting the first microfluidic channel and the collection channel may be no more than 500, no more than 100, no more than 50, no more than 20, no more than 10, no more than 5, no more than 3, no more than 2, or no more than 1. Combinations of these ranges are possible. In some cases, there may be, e.g., at least 2, at least 3, at least 4, at least 5 of the second channels, and optionally no more than 10, no more than 9, no more than 8, no more than 7, no more than 6, or no more than 5 of the second channels. In some cases, the plurality of the second channels are distributed (e.g., evenly) over a distance of no more than 10 μm, no more than 9 μm, no more than 8 μm, no more than 7 μm, no more than 6 μm, no more than 5 μm, no more than 4 μm, no more than 3 μm, no more than 2 μm, or no more than 1 μm. In certain embodiments, each adjacent pair of the plurality of the second channels is spaced apart by a distance of no more than 5 μm, no more than 4 μm, no more than 3 μm, no more than 2 μm, no more than 1 μm, no more than 0.75 μm, no more than 0.5 μm, or no more than 0.25 μm.

In some cases, the first channels and the second channels can be non-parallel with each other. In some cases, the second channels are arranged at an angle relative to the first channels in a range of from 0 to 180 degrees (e.g., from 5 to 175 degrees, from 10 to 170 degrees, from 15 to 165 degrees, from 20 to 160 degrees, from 25 to 155 degrees, from 30 to 150 degrees, from 35 to 145 degrees, from 40 to 140 degrees, from 45 to 135 degrees, from 50 to 130 degrees, from 55 to 125 degrees, from 60 to 120 degrees, from 65 to 115 degrees, from 70 to 110 degrees, from 75 to 105 degrees, from 80 to 100 degrees) (e.g., wherein the second channels are arranged perpendicular to the first channels). In some embodiments, for each of the first channels, the at least one of the second channels are arranged at an angle relative to the first channel in a range of from 0 to 180 degrees (e.g., from 5 to 175 degrees, from 10 to 170 degrees, from 15 to 165 degrees, from 20 to 160 degrees, from 25 to 155 degrees, from 30 to 150 degrees, from 35 to 145 degrees, from 40 to 140 degrees, from 45 to 135 degrees, from 50 to 130 degrees, from 55 to 125 degrees, from 60 to 120 degrees, from 65 to 115 degrees, from 70 to 110 degrees, from 75 to 105 degrees, from 80 to 100 degrees) (e.g., wherein the second channels are arranged perpendicular to the first channels).

In some cases, for each of the first channels, the plurality of the second channels are disposed (e.g., spaced and/or distributed) to apply a pressure gradient at least two distinct locations on a biological component corresponding in size to the first channel when the biological component is disposed in the first channel. In some embodiments, for each of the first channels, the at least one of the second channels are disposed (e.g., spaced and/or distributed) relative to the first channel to apply a pressure gradient to a side of a biological component when disposed in the first channel.

In some cases, the first channels and the second channels are arranged (e.g., sized, shaped, and/or oriented) to trap (e.g., removably trap) one or more infectious agents (e.g., one or more viruses, one or more bacteria, one or more fungi, one or more prokaryotic cells, one or more eukaryotic cells, or a combination thereof), or other second entities, in and/or at the first channels. In certain cases, the first channels and the second channels are arranged (e.g., sized, shaped, and/or oriented) to trap (e.g., removably trap) one or more infectious agents (e.g., one or more viruses, one or more bacteria, one or more fungi, or a combination thereof), or other second entities, in and/or at the first channels. In some cases, the first channels and the second channels are arranged (e.g., sized, shaped, and/or oriented) to lyse one or more cellular biological components (e.g., blood cells and/or epithelial cells), or other second entities, with the first channels and the second channels.

In some embodiments, a microfluidic filter comprises a first channel and one or more second channels directly fluidically connected to the first channel downstream of the first channel. In some embodiments, a microfluidic filter comprises a first channel and one or more second channels physically connected to the first channel. In some embodiments, such one or more second channels comprises a second channel that is directly fluidically connected to a third channel disposed downstream of the second channel. In some embodiments, such one or more second channels comprises a second channel that is physically connected to a third channel. In some embodiments, such one or more second channels (e.g., further) comprises a second channel that is directly fluidically connected to a common fluid collection channel disposed downstream of the second channel. In some embodiments, such one or more second channels (e.g., further) comprises a second channel that is physically connected to a common fluid collection channel. In some embodiments, a microfluidic filter comprises second channels where different ones of the second channels are oriented in different directions (e.g., one or more second channels disposed perpendicular to one or more other second channels). FIGS. 28A-B illustrate an example of microfluidic filters each comprising second channels fluidically connected to a first channel downstream of the first channel where the second channels comprise (i) a set of second channels directly fluidically connected to a third channel disposed downstream of the set of second channels and physically connected to the third channel and (ii) a second channel that is directly fluidically connected to a common fluid collection channel disposed downstream of the second channel and physically connected to the common fluid collection channel.

In some embodiments, a separation unit comprises a first channel and one or more second channels fluidically connected to the first channel downstream of the first channel. In some embodiments, a separation unit comprises a first channel and one or more second channels physically connected to the first channel. In some embodiments, such one or more second channels comprises a second channel that is directly fluidically connected to a third channel disposed downstream of the second channel. In some embodiments, such one or more second channels comprises a second channel that is physically connected to a third channel. In some embodiments, such one or more second channels (e.g., further) comprises a second channel that is directly fluidically connected to a common fluid collection channel disposed downstream of the second channel. In some embodiments, such one or more second channels (e.g., further) comprises a second channel that is physically connected to a common fluid collection channel. In some embodiments, a separation unit comprises second channels where different ones of the second channels are oriented in different directions (e.g., one or more second channels disposed perpendicular to one or more other second channels). FIGS. 28A-B illustrate an example of separation units each comprising second channels fluidically connected to a first channel downstream of the first channel where the second channels comprise (i) a set of second channels directly fluidically connected to a third channel disposed downstream of the set of second channels and physically connected to the third channel and (ii) a second channel that is directly fluidically connected to a common fluid collection channel disposed downstream of the second channel and physically connected to the common fluid collection channel.

In some embodiments, a second channel in a microfluidic filter is directly fluidically connected to a common fluid collection channel. In some embodiments, a second channel in a microfluidic filter is physically connected to a common fluid collection channel. In some embodiments, a second channel in a separation unit is directly fluidically connected to a common fluid collection channel. In some embodiments, a second channel in a separation unit is physically connected to a common fluid collection channel.

The microfluidic device may have a plurality of repeat units. The repeat units may be arranged in any arrangement within a microfluidic device, for example, in a parallel arrangement.

Some or all of the repeat units may comprise a first microfluidic channel and a second microfluidic channel, and a plurality of substantially parallel channel connecting the first microfluidic channel and the second microfluidic channel. In some cases, the repeat units are uniform in arrangement (e.g., are identically arranged as each other), and/or the repeat the units may be uniform in size (e.g., wherein the channels comprised in each of the units are sized identically). In some cases, the repeat units may define biological component separation units or biological component lysing units. For example, as discussed herein, the channels may be arranged (e.g., sized, shaped, and/or oriented) such that fluid flow through the units causes selective lysing of one or more biological components.

According to some embodiments, a microfluidic device may have at least 1,000 repeat units, at least 3,000 repeat units, at least 10,000 repeat units, at least 30,000 repeat units, at least 50,000 repeat units, at least 100,000 repeat units, at least 200,000 repeat units, at least 300,000 repeat units, at least 400,000 repeat units, at least 500,000 repeat units, at least 1,000,000 repeat units. According to some embodiments, a microfluidic device may have no more than 100,000 repeat units, no more than 30,000 repeat units, no more than 10,000 repeat units, no more than 3,000 repeat units, or no more than 1,000 repeat units. Combinations of these ranges are possible. The repeat units may be arranged in a regular array (e.g., a 1D or 2D array).

The repeat units may be identical or substantially similar, according to some embodiments. Without wishing to be bound by theory, it may be advantageous to have identical repeat units. One or more of the repeat units may have a particular configuration (e.g., a certain angle of the first microfluidic channel and second microfluidic channel) that is substantially similar.

Certain microfluidic channels (e.g., first microfluidic channel, second microfluidic channel, or third microfluidic channel) may have a geometry. The microfluidic channels may be substantially linear, curvilinear, non-linear, or a combination thereof. For example, one set of embodiments, the first microfluidic channels are substantially linear and at least some of the second microfluidic channels are curvilinear.

In some cases, the first channels and the second channels are sized and arranged such that at least 70% (e.g., at least 80%, at least 90%, at least 95%, at least 99%) (e.g., all) bacteria in fluid that is flowed through the first channels and the second channels are separated (e.g., are removably trapped at or in the device (e.g., at or in the first channels or the second channels) (e.g., at or in the first channels)).

Some or all of the repeat units may have microchannels that are substantially parallel. For example, one or more of the second microfluidic channels may be substantially parallel to each other. In certain embodiments, some microfluidic channels, such as second microfluidic channels, may substantially identical. However, in some embodiments, the microfluidic channels may not be substantially parallel. For example, the microfluidic channels may have a different direction or orientation, such as those described later herein.

The microfluidic device may have an inlet and an outlet. In certain cases, the inlet and outlet may be in fluid communication with the plurality of repeat units, which may be used in a variety of ways. In one example, a sample may be inserted through the inlet and a portion of the sample (e.g., cell debris in buffer) is collected through the outlet. In another example, a sample having particles and mammalian cells is inserted through the inlet, and lysed mammalian cells and cell components are collected through the outlet. In certain embodiments, a sample (e.g., fluid comprising a plurality of cells) is flowed through a microfluidic channel using a syringe. In some embodiments, fluid may flow through the device at a rate of at least 0.01 mL/min (e.g., at least 1 mL/min, at least 5 mL/min, at least 10 mL/min, at least 20 mL/min, at least 25 mL/min, at least 50 mL/min, at least 100 mL/min, or at least 200 mL/min). In some cases, fluid may be provided, e.g., from a syringe, to the device (e.g., at an inlet thereof) thereby causing the fluid to flow through the device. In some embodiments, the method is performed using a volume of fluid of at least 0.5 mL (e.g., at least 1 mL, at least 2 mL, at least 10 mL, at least 25 mL, at least 50 mL, at least 75 mL, at least 100 mL, at least 200 mL, at least 500 mL, at least 750 mL, at least 1 L, at least 2 L, at least 3 L, at least 4 L, at least 5 L, at least 10 L), preferably over a period of no more than 2 h, no more than 1 h, no more than 30 min, no more than 20 min, no more than 10 min, no more than 5 min, no more than 2 min, or no more than 1 min).

Certain embodiments include a flow path through the microfluidic device. The flow path (e.g., flow path between inlet and outlet) may pass through a repeat unit of the plurality of repeat units. In some cases, the flow path may pass through only one repeat unit of the plurality of repeat units.

The microfluidic device may have one or more flow channels. The flow channels may be in fluid communication with the inlet and/or repeat units. The flow channels may be configured to receive a sample (e.g., blood sample) having a plurality of cells. One or more waste channels in fluid communication with the outlet may be present. The waste channels may be in configuration with the outlet and/or repeat units. The flow channel and the waste channel may be in fluid communication (e.g., though second microfluidic channels). In certain cases, some cells (e.g., mammalian cells) in a sample may be lysed before reaching the waste channel, such that the waste channel is configured to receive lysed cell components. In some aspects, the waste channel may be free of second type of cells (e.g., fungal cells) and/or particles.

A microfluidic channel within the microfluidic device may have a flared entrance, in certain embodiments. The flared entrance may be a linear or a curved flared opening, as examples. The flared entrance may allow certain cells to be gradually distorted as they flow through the channel. For example, a mammalian cells may enter a first fluid channel with a flared entrance having a shape similar to a perfect sphere and may be distorted to have an aspect ratio of at least 2:1 as it flows through the first fluidic channel.

Certain aspects of the present disclosure describe a direction (e.g., first direction, second direction) where cells can be flowed within a microfluidic device. The first direction and the second direction may define an angle of at least 30°, at least 35°, at least 40°, at least 50°, at least 55°, or at least 60°. The first direction and the second direction may define an angle of no more than 60°, no more than 55°, no more than 50°, no more than 45°, no more than 40°, no more than 35°, or no more than 30°. Combinations of these ranges are possible. In some embodiments, the first direction and the second direction define an angle between 0° and 180°, between 10° and 170°, between 20° and 160°, between 30° and 150°, between 40° and 140°, between 50° and 130°, between 60° and 120°, between 70° and 110°, between 80° and 100°, or 90°.

In certain embodiments, a microfluidic channel (e.g., a first microfluidic channel, a second microfluidic channel, or a third microfluidic channel) may have a maximum cross-sectional dimension (e.g., diameter) that is no more than 100 micrometers, no more than 10 micrometers, no more than 5 micrometers, no more than 1 micrometers, no more than 750 nanometers, no more than 500 nanometers, no more than 250 nanometers, or no more than 100 nanometers. In certain embodiments, the microfluidic channel may have a maximum cross-sectional dimension that is no more than 1 micrometer, no more than 5 micrometers, no more than 10 micrometers, or no more than 100 micrometers. In certain embodiments, the microfluidic channel may have a maximum cross-sectional dimension that is at least 50 nanometers, at least 100 nanometers, at least 250 nanometers, at least 500 nanometers, at least 750 nanometers, at least 1 micrometer, at least 5 micrometers, at least 10 micrometers, or at least 100 micrometers. Combinations of these ranges are possible.

In certain embodiments, the second microfluidic channel may have a maximum cross-sectional dimension (e.g., diameter, area, height, or width) that is no more than 10 micrometers, no more than 1 micrometers, no more than 0.5 micrometers, or no more than 0.1 micrometers. In certain embodiments, the confining microfluidic channel may have a maximum cross-sectional dimension (e.g., diameter, area, height, or width) that is no more than 0.1 micrometer, no more than 0.5 micrometers, no more than 1 micrometers, or no more than 10 micrometers. In certain embodiments, the confining microfluidic channel may have a maximum cross-sectional dimension (e.g., diameter, area, height, or width) that is at least 0.1 micrometer, at least 0.5 micrometers, at least 1 micrometers, or at least 10 micrometers. Combinations of these ranges are possible.

In some embodiments, the first microfluidic channel has a maximum cross-sectional diameter that is larger that the maximum cross-sectional diameter of the second microfluidic channel.

In certain embodiments, the ratio of a cross-sectional dimension of a first microfluidic channel to a cross-sectional dimension of a second microfluidic channel is at least 0.001, at least 0.01, at least 0.1, at least 1, at least 10, at least 100, or at least 1000. In certain embodiments, the ratio of a cross-sectional dimension of a first microfluidic channel to a cross-sectional dimension of a second microfluidic channel is at no more than 1000, no more than 100, no more than 10, no more than 1, no more than 0.1, no more than 0.01, or no more than 0.001. In certain cases, the ratio of a cross-sectional dimension of a first microfluidic channel to a cross-sectional dimension of a second microfluidic channel is between 0.001 and 1000, between 0.01 and 100, or between 0.1 and 10. The cross-sectional dimension may be, for example, an area, a diameter, a height, or a width,.

The microfluidic channels within a microfluidic device may have a suitable length ratio. In certain embodiments, the ratio of a length of a first microfluidic channel to a length of a second microfluidic channel is at least 0.001, at least 0.01, at least 0.1, at least 1, at least 10, at least 100, or at least 1000. In certain embodiments, the ratio of a length of a first microfluidic channel to a length of a second microfluidic channel is at no more than 1000, no more than 100, no more than 10, no more than 1, no more than 0.1, no more than 0.01, or no more than 0.001. In certain cases, the ratio of a length of a first microfluidic channel to a length of a second microfluidic channel is between 0.001 and 1000, between 0.01 and 100, or between 0.1 and 10.

The microfluidic channels within a microfluidic device may have a suitable volume ratio. In certain embodiments, the ratio of volume of a first microfluidic channel to a volume of a second microfluidic channel is at least 0.001, at least 0.01, at least 0.1, at least 1, at least 10, at least 100, or at least 1000. In certain embodiments, the ratio of a volume of a first microfluidic channel to a volume of a second microfluidic channel is at no more than 1000, no more than 100, no more than 10, no more than 1, no more than 0.1, no more than 0.01, or no more than 0.001. In certain cases, the ratio of a volume of a first microfluidic channel to a volume of a second microfluidic channel is between 0.001 and 1000, between 0.01 and 100, or between 0.1 and 10.

In certain embodiments, the ratio of a cross-sectional dimension of a first microfluidic channel to a cross-sectional dimension of a collection channel is at least 0.001, at least 0.01, at least 0.1, at least 1, at least 10, at least 100, or at least 1000. In certain embodiments, the ratio of a cross-sectional dimension of a first microfluidic channel to a cross-sectional dimension of a collection channel is at no more than 1000, no more than 100, no more than 10, no more than 1, no more than 0.1, no more than 0.01, or no more than 0.001. In certain cases, the ratio of a cross-sectional dimension of a first microfluidic channel to a cross-sectional dimension of a collection channel is between 0.001 and 1000, between 0.01 and 100, or between 0.1 and 10. The cross-sectional dimension may be, for example, an area, a diameter, a height, or a width.

The microfluidic channels within a microfluidic device may have a suitable length ratio. In certain embodiments, the ratio of a length of a first microfluidic channel to a length of a collection channel is at least 0.001, at least 0.01, at least 0.1, at least 1, at least 10, at least 100, or at least 1000. In certain embodiments, the ratio of a length of a first microfluidic channel to a length of a collection channel is at no more than 1000, no more than 100, no more than 10, no more than 1, no more than 0.1, no more than 0.01, or no more than 0.001. In certain cases, the ratio of a length of a first microfluidic channel to a length of a collection channel is between 0.001 and 1000, between 0.01 and 100, or between 0.1 and 10.

The microfluidic channels within a microfluidic device may have a suitable volume ratio. In certain embodiments, the ratio of volume of a first microfluidic channel to a volume of a collection channel is at least 0.001, at least 0.01, at least 0.1, at least 1, at least 10, at least 100, or at least 1000. In certain embodiments, the ratio of a volume of a first microfluidic channel to a volume of a collection channel is at no more than 1000, no more than 100, no more than 10, no more than 1, no more than 0.1, no more than 0.01, or no more than 0.001. In certain cases, the ratio of a volume of a first microfluidic channel to a volume of a collection channel is between 0.001 and 1000, between 0.01 and 100, or between 0.1 and 10.

The microfluidic device may comprise glass. In some cases, any microfluidic channel within the microfluidic device may be defined by the glass (e.g., microfluidic glass walls). The repeat units may also be defined by the wall. In certain cases, the microfluidic device may be made of other material such as, for example, silicon or a polymer (e.g., polymethyl methacrylate). For example, in one set of embodiments, the repeat units within the microfluidic device are defined by a polymer (e.g., polymer microfluidic walls). In some cases, the microfluidic channels may be disposed in a patterned layer (e.g., an elastomer layer) (e.g., a microfluidic layer) (e.g., bounded by plastic or glass (e.g., a coverslip)). In some cases, the device may be transparent, or some or all of the channels (e.g., the first channels and the second channels) may be visible through the device.

In certain aspects, the first microfluidic channel has a first Reynolds Number, and the fluid in the second microfluidic channel has a second Reynolds Number different from the first Reynolds Number.

The microfluidic devices disclosed herein may be a removable cartridge, according to some embodiments.

In some aspects, flowing a fluid through the microfluidic device may not clog the microfluidic device. It has been appreciated that flowing a sample (e.g., blood sample) may not clog the microfluidic device. In certain embodiments, the microchannels (e.g., first microfluidic channel, second microfluidic channel) are substantially free of clogging components (e.g., cell debris) after flowing a fluid through the device. For example, according to some embodiments, a microfluidic device may have a portion of microfluidic channels that not clogged of at least 80%, at least 85%, at least 90%, at least 95%, or at least 100% of the microfluidic channels.

Certain embodiments of the present disclosure are directed to various articles, systems, and methods that may reduce or mitigate the need to pretreat a sample. For example, if certain cells within a sample are lysed in a microfluidic device, centrifugation of the sample prior to being inserted to the microfluidic device may not be necessary. In certain aspects, the present disclosure may use samples “as-is” (e.g., without pretreatment or any prior step) after obtaining the sample. For example, a blood sample is withdrawn from a subject and then is flowed through a microfluidic device described herein.

Some embodiments are directed to a first type of cell and/or a second type of cell (or other second entity). In some aspects, the first type of cells may have an average volume that is bigger than the second type of cells. For example, in some embodiments, the average volume of a first type of cells may be bigger by less than a 2-fold, less than a 3-fold, less than a 4-fold, less than a 5-fold, less than a 10-fold, or less than a 20-fold than the average volume of a second type of cells. In yet some other embodiments, the average volume of the first type of cells is bigger by no more than a 20-fold, no more than a 10-fold, no more than a 5-fold, no more than a 4-fold, no more than a 3-fold, or no more than a 2-fold. Combinations of the ranges are possible.

The first type of cell may have mechanical properties (e.g., surface mechanical properties) that are different from mechanical properties of the second type of cells, or other second entities. In certain cases, the mechanical properties of the first type of cells may cause the cells to be more easily distortable than the second type of cells. In some cases, the first type of cells may be more easily lysed when flowed through the microfluidic channel, which may be attributed in part to their mechanical properties. Some cells of the first type of cells may have mechanical properties that allow their membrane to be ruptured, according to some embodiments.

According to certain embodiments, the first type of cells may pass therethrough (e.g., through a second microfluidic channel) and the second type of cell or other second entity may not pass therethrough. In some embodiments, the second type of cells may get stuck in some microfluidic channels of a plurality of microfluidic channels. It may be advantageous for the second type of cells or other second entities to get stuck or trapped for various reasons (e.g., to be analyzed, separated, or further processed). In some cases, as discussed herein, the second type of cells or entities may be backflushed (e.g., flowed back towards the inlet) to be removed from the device, and in some cases, analyzed. However, in other embodiments, the second type of cells or entities may be analyzed while still in the microfluidic device.

The first type of cell may be of a suitable type. Non-limiting examples of the first type of cell include blood cells (e.g., red blood cells, white blood cells, or platelets), fibroblasts, epithelial cells, lymphocytes (e.g., blood cells), macrophages, neurons (e.g., nerve cells), muscle cells (e.g., cardiac cells), adipocytes (e.g., fat cells), endothelial cells, or tumor cells (e.g., circulating tumor cells). The first type of cell may comprise mammalian cells. The mammalian cells may be obtained from a human or non-human mammal, such as a monkey, cow, sheep, goat, horse, rabbit, pig, mouse, rat, dog, or cat.

The second type of cells may comprise microbial cells (e.g., bacteria, fungi, viruses, parasites). Non-limiting examples of bacteria cells include Escherichia coli, Staphylococcus spp., Klebsiella spp., Acinetobacter spp., Pseudomonas spp., or Enterococcus spp. The bacterial cells may be gram-negative or gram-positive. Non-limiting examples of fungal cells include yeast (e.g., Candida spp), hyphae cells, and chytrid cells. Non-limiting examples of parasites include Giardia lamblia (e.g., Giardia intestinalis), Cryptosporidium parvum, Entamoeba histolytica (e.g., amoeba), Toxoplasma gondii, and Plasmodium falciparum (e.g., malaria). Some non-limiting examples of viruses include norovirus, herpes virus, dengue virus, parvoviridae virus, caliciviradae virus, poxvirus, and human immunodeficiency virus.

Thus, the second entity may be a cell in accordance with one set of embodiments. However, the second entity need not be a cell, and may be another entity in other embodiments, for example, a particle or a virus. In some cases, the second entities may be particles, bacteria, parasitic cells, Plasmodium cells, exosomes, viruses, extracellular vesicles, or fungi (e.g., yeast). In certain embodiments, the second entities may be determined through applying multi-omic analysis to the second entities.

According to one set of embodiments, a first type of cells suspected of containing therein a second type of cells through a microfluidic filter (e.g., microfluidic channel) such that the first type of cells are lysed and the second type of cells are trapped by the microfluidic filter. For example, a human blood cell suspected of containing a virus may be flowed through a microfluidic device, such that the human blood cell may be lysed, and the virus may get stuck in the microfluidic device. The microfluidic device may then be analyzed to determine infection of the cells by the virus.

The sample having the plurality of cells may be of a suitable type and may have other components. The sample may be a fluid (e.g., water, cell media) and may include, for example, one or more buffers and/or one or more lysing agents. The sample may be an aqueous sample or may have different phases. The fluid sample may be obtained from a subject (e.g., human subject). The fluid (e.g., fluid sample) may comprise blood, urine, sputum, stool, biopsy, exudate (e.g., pus), bodily discharge, skin swab, wound swab, mucus, cerebrospinal fluid, lymph, tissue, breast milk, fluid aspirate, wound drainage, abscess, food, juice, or wastewater. The fluid may be, or may be derived from, blood (e.g., whole or dilute), urine, stool, lymph, tissue, nasal or cheek swab, mucus, saliva, sputum, cerebrospinal fluid, breast milk, fluid aspirate, or wound or abscess drainage. In some cases, the sample having the plurality of cells may not be from a subject. For example, the fluid may be a saline sample, a soil sample, a rainwater sample, a food sample. The fluid may be aqueous. As non-limiting examples, the fluid may be wastewater, a pharmaceutical composition (e.g., a fluid comprising a drug or biologic), or the fluid may be or may be derived from food, drinking water, or a beverage. The sample may be an extract from a plant or animal in some cases.

Accordingly, in some embodiments, the device may be operable to separate (e.g., isolate) one or more biological components in a fluid (e.g., blood, urine, saliva, sputum) [e.g., from one or more other biological components (e.g., cells) in the fluid], and/or the device may be operable to selectively lyse one or more biological components [e.g., cells (e.g., blood cells)] in a fluid (e.g., blood, urine, saliva, sputum). For example, the device may be operable to selectively lyse the one or more biological components while preserving one or more other biological components [e.g., one or more infectious agents (e.g., one or more viruses, one or more bacteria, one or more fungi, or a combination thereof)] in the fluid.

The biological components (e.g., the biological component) may include one or more infectious agents (e.g., one or more viruses, one or more bacteria, one or more fungi, or a combination thereof). In some cases, the biological components (e.g., the biological component) may comprise one or more cellular components. Other examples of biological components, or other second entities, are discussed in more detail herein.

In some embodiments, in addition to cells, the sample may have other components such as, for example, particles, The particles in the sample may not pass through some of the microfluidic channels (e.g., second microfluidic channel) of the microfluidic device. The particles may have a size (e.g., average diameter) that is larger than the size (e.g., cross-sectional diameter) of a microfluidic channel. In some cases, the cells (e.g., first type of cells) may have mechanical properties that allow distortion (e.g., squeezing cells), whereas the particles may not be distorted in a microfluidic channel.

The blood sample may be of a suitable type. For example, according to some embodiments, the blood sample may be a whole blood sample, fresh blood sample, (e.g., prior to being flowed), a frozen blood sample frozen, a blood sample with an anticoagulant (e.g., ethylenediaminetetraacetic acid (EDTA)), and/or an untreated blood sample. In one set of embodiments, the blood sample comprises a second type of cells, where the second type of cells are suspected of causing sepsis in a subject. The blood sample may comprise red blood cells (e.g., sickle red blood cells) and/or white blood cells.

In some embodiments, a composition derived from tissue samples, wherein a composition comprises viable target cancer cells, bacteria, parasite, virus, and/or fungi and is substantially devoid of healthy parenchyma cells (e.g., hepatocytes in the case of liver tissue, astrocytes and neurons in the case of brain tissue) (e.g., such that culturing and/or identification and/or testing can be performed using a microfluidics device).

In some embodiments, the sample may have a volume of at least 0.01 mL, at least 0.1 mL, at least 1 mL, at least 10 mL, or at least 100 mL. In some embodiments, the sample may have a volume of no more than 100 mL, no more than 10 mL, no more than 1 mL, no more than 0.1 mL, or no more than 0.01 mL. Combinations of these ranges are possible.

In some embodiments, the sample may be sonicated prior to being flowed through the microfluidic device.

In some embodiments, the microfluidic device is configured to operate with singular or multimodal microscopy imaging to identify, characterize and/or track any of the cells within the microfluidic device while or after flowing the cells. For example, in some cases, the cells may be imaged while a fluid is being flowed through the device. However, in some other cases, the cells may be imaged after trapping some cells within the microfluidic device. Some non-limiting examples of techniques include brightfield microscopy, reconstructed quantitative phase microscopy (QPM) images, hyperspectral imaging, Raman Spectroscopy, and instant Structured Illumination Microscopy. In some embodiments, for at least one field of view (FOV), the image captured is a combination of at least one of the imaging modalities selected from brightfield, hyperspectral, Raman, iSIM or other microscopy techniques.

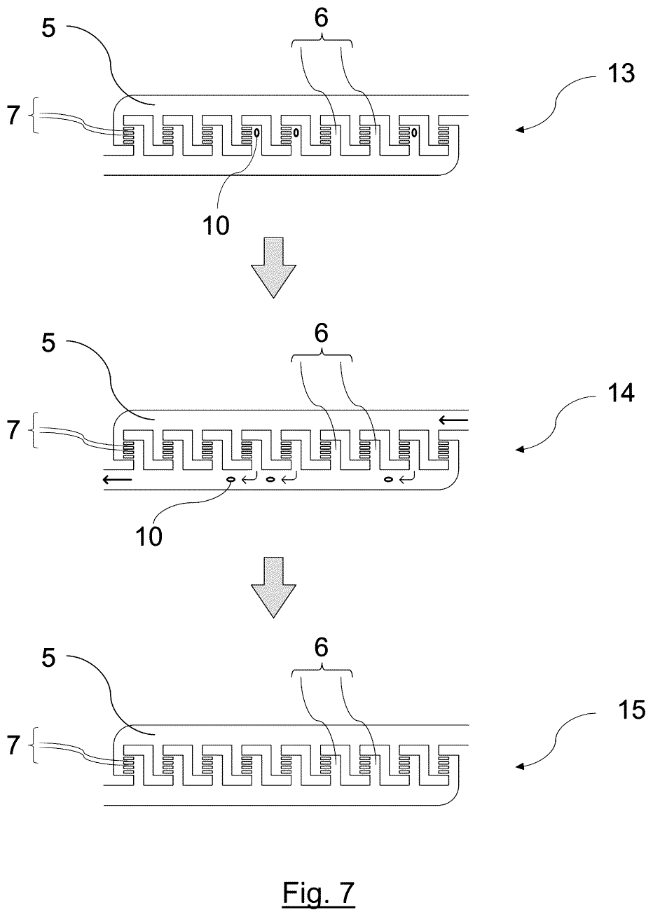

After flowing a plurality of cells, certain cells (e.g., second type of cells) may not flow through a microfluidic channel and may be collected. For example, it may be possible to flow a plurality of cells having mammalian cells and fungal cells, lysing the mammalian cells while retaining the fungal cells, and collecting the fungal cells. In one embodiment, a fluid flows through the outlet, such that cells stuck (e.g., in a flow channel) may be released from the microfluidic device and collected as a sample. In certain aspects, the sample collected (e.g., cells that are released from the microfluidic channel) is substantially purer (e.g., less components) than the sample that was initially flowed through the microfluidic device. Thus, in some embodiments, the microfluidic device described can filter out certain components. As shown in FIG. 7, cell 10 of a second type of cells that is stuck in a microfluidic channel flow out of a microchannel. In certain cases, cell stuck in a microchannel (e.g., second microfluidic channel) may be released by flowing a fluid in an opposite direction (e.g., reverse flow) of the direction where the fluid was initially flowed. For example, a plurality of cells having bacterial cells may be flowed through the inlet of a microfluidic device in a direction, such that some bacterial cells get stuck in the microchannels, and a fluid (e.g., buffer) may flow through the outlet of the device in an opposite direction such that the bacterial cells are released. For example, certain embodiments are generally directed to removing at least a portion (e.g., at least 50%, at least 60%, at least 70%, at least 80%, at least 90%, at least 95%, or at least 99%) of the at least 50% of the first biological component or other second entity in the fluid from the channels (e.g., by reversing a direction of fluid flow)

A purified sample (e.g., filtered second type of cells) can be collected and analyzed using one or more techniques, such as, for example, polymerase chain reaction, plasmid transformation, extraction (e.g., DNA extraction), fluorescence in situ hybridization, or microscopy. In certain embodiments, the second type of cells may be identified, sequenced, genotyped, or fluorescently labeled.

In some aspects of the present disclosure, the cells stuck in the microfluidic device may be further analyzed. In one example, antibiotic susceptibility testing (AST) may be performed in the microfluidic device to determine the susceptibility to antimicrobials of bacterial cells stuck in the microfluidic channels. In another example, a microscope can image the cells present in the microfluidic channels.

The present disclosure describes several methods, where such method can have a particular use. However, it is to be understood that such methods of use are not exclusive. In one set of embodiments, the method is a method of determining cancer in a subject. In another set of embodiments, the method is a method of determining a disease in a subject. In yet another set of embodiments, the method is a method of determining a genetic disorder in a subject. In certain embodiments, the method is a method of determining a parasite in a subject. In some embodiments, the method is a method of determining an infection (e.g., bacterial infection and/or viral infection) in a subject.

A lysing composition may be included in certain embodiments. The lysing composition may comprise a variety of suitable components. The lysing composition may affect the elasticity of some cells (e.g., mammalian cells). Non-limiting examples of some components in a lysing composition include a lysing agent, a detergent, a surfactant, nonionic agents (e.g., a nonionic surfactant), non-denaturing agents, saponin, Triton X-100, IGEPAL CA-630, ammonium chloride, sodium chloride, sodium chloride, water, glycerol, Iodixanol, and/or phosphate buffered saline (PBS) microorganism growth media. In one embodiment, the detergent is a saponin. In some cases, the detergent is provided before the fluid is provided to the device (e.g., by mixing in a syringe (e.g., immediately) prior to providing the fluid). In some cases, the detergent is provided to the fluid in the device.

The lysing composition may be mixed with a sample (e.g., blood sample). In some embodiments, a composition is formed from a combination of a lysing composition comprising lysing agent and a biological sample (e.g., blood sample).

In some embodiments, contacting a blood sample within a microfluidic device to a lysing composition comprises rocking a sample and a lysing composition together beforehand. Rocking may occur from a period of time of at least 1 min and no more than 10 min. In some embodiments, a rocking occurs from a period of time of at least 3 min and no more than 7 min. In some embodiments, a rocking occurs from a period of time of at least 10 seconds and no more than 120 seconds.

In some embodiments, contacting a blood sample within a microfluidic device to a lysing composition comprises vortexing a sample and a lysing composition together beforehand. In some embodiments, vortexing occurs from a period of time of at least 10 sec and no more than 1 min. In some embodiments, vortexing occurs from a period of time of at least 20 sec and no more than 40 sec.

In some embodiments, a lysing composition is a portion of a stock solution in the assay. In some embodiments, a lysed blood composition is a solution.

In some embodiments, a step of contacting a blood sample and a lysing composition within a microfluidic device comprises combining a blood sample and a lysing composition in a ratio in a range of from 30:1 to 1:9 (e.g., 1:1 or 24:1). In some embodiments, a ratio is in a range of from 1:1 to 1:5. In some embodiments, a ratio is in a range of from 22:1 to 26:1. In some embodiments, a step of contacting a blood sample and a lysing composition within a microfluidic device comprises combining a blood sample and a lysing composition such that a blood sample is at least 10% (e.g., at least 10%, at least 50%, at least 90%, or at least 95%) of total volume thereof. In some embodiments, a step of contacting a blood sample and a lysing composition within a microfluidic device comprises combining a blood sample and a lysing composition in a ratio such that lysing agent from a lysing composition is present in a concentration of at least 0.4% and no more than 3% (e.g., by volume). In some embodiments, a step of contacting a blood sample and a lysing composition within a microfluidic device comprises combining a blood sample and a lysing composition in a ratio such that lysing agent from a lysing composition is present in a concentration of at least 0.5% and no more than 1.5% (e.g., by volume). In some embodiments, a step of contacting a blood sample and a lysing composition within a microfluidic device comprises combining a blood sample and a lysing composition in a ratio such that lysing agent from a lysing composition is present in a concentration of at least 0.75% and no more than 1.25% (e.g., by volume). In some embodiments, a step of contacting a blood sample and a lysing composition within a microfluidic device comprises combining a blood sample and a lysing composition such that a lysing composition is no more than 90% (e.g., no more than 90%, no more than 50%, no more than 20%, or no more than 5%) of total volume thereof.

In some embodiments, a method includes culturing a target human cell, a bacteria, parasites, viruses, and/or fungi from a lysed blood composition (e.g., within 60 min, within 30 min, within 15 min, or within 10 min of a contacting). In some embodiments, a culturing is performed using a microfluidics device. In some embodiments, a microfluidic device comprises multi-cellular growth chambers for expanding the enriched target human, and/or non-human pathogen populations in cell groups and/or clusters. In some embodiments, a microfluidics device comprises single-cell-sized growth trenches and a culturing is performed using trenches.

In some embodiments, a method includes, after a microfluidic contacting, quenching a sample with a lysis-ceasing composition. In some embodiments, a lysis-ceasing composition is phosphate buffered saline. In some embodiments, the quenching sample flows through the microfluidic device and is followed by an alternate buffer and/or reagent.

In some embodiments, a concentration of lysing agent in a lysing composition is at least 10% (e.g., by volume). In some embodiments, a concentration of lysing agent in a lysing composition is at least 15% (e.g., by volume). In some embodiments, a concentration of lysing agent in a lysing composition is at least 20% (e.g., by volume). In some embodiments, a concentration of lysing agent in a lysing composition is at least 25% (e.g., by volume). In some embodiments, a concentration of lysing agent in a lysing composition is no more than 50% (e.g., by volume). In some embodiments, a concentration of lysing agent in total volume of a lysing composition and a blood sample is at least 0.4% and no more than 3% (e.g., by volume). In some embodiments, a concentration of lysing agent in total volume of a lysing composition and a blood sample is at least 50% and no more than 90% (e.g., by volume). In some embodiments, a concentration of lysing agent in total volume of a lysing composition and a blood sample is at least 0.75% and no more than 1.25% (e.g., by volume). In some embodiments, a concentration of lysing agent in total volume of a lysing composition and a blood sample is 1% (e.g., by volume). In some embodiments, a concentration of lysing agent in a formed composition is at least 0.4% and no more than 3% (e.g., by volume). In some embodiments, a concentration of lysing agent in a formed composition is at least 0.5% and no more than 1.5% (e.g., by volume). In some embodiments, a concentration of lysing agent in a formed composition is at least 0.75% and no more than 1.25% (e.g., by volume). In some embodiments, a concentration of lysing agent in a formed composition is 1% (e.g., by volume).

In some embodiments, a composition has been formed from a combination of a blood sample and a lysing composition within a microfluidic device in a ratio in a range of from 30:1 to 1:9 (e.g., 1:1 or 24:1). In some embodiments, a ratio is in a range of from 1:1 to 1:5. In some embodiments, a ratio is in a range of from 22:1 to 26:1. In some embodiments, a step of contacting a blood sample and a lysing composition comprises combining a blood sample and a lysing composition such that a blood sample is at least 10% (e.g., at least 10%, at least 50%, at least 90%, or at least 95%) of total volume thereof. In some embodiments, a composition has been formed from a combination of a blood sample and a lysing composition in a ratio such that lysing agent from a lysing composition is present in a concentration of at least 0.4% and no more than 3% (e.g., by volume). In some embodiments, a composition has been formed from a combination of a blood sample and a lysing composition in a ratio such that lysing agent from a lysing composition is present in a concentration of at least 0.5% and no more than 1.5% (e.g., by volume). In some embodiments, a composition has been formed from a combination of a blood sample and a lysing composition in a ratio such that lysing agent from a lysing composition is present in a concentration of at least 0.75% and no more than 1.25% (e.g., by volume). In some embodiments, a composition has been formed from a combination of a blood sample and a lysing composition such that a lysing composition is no more than 25% (e.g., no more than 20%, no more than 15%, no more than 10%, or no more than 5%) of total volume thereof.

Devices (e.g., microfluidic devices) disclosed herein may have high trapping efficiencies, high separation efficiencies, high retrieval efficiencies, high lysing efficiency, and/or low detection limits for one or more biological components of interest. A device may have a trapping efficiency of at least 50%, at least 60%, at least 70%, at least 80%, at least 90%, at least 95%, at least 97%, at least 99%, at least 99.5%, at least 99.9%, at least 99.99%, at least 99.9999%, at least 99.999999%, or 100% for a biological component (e.g., a bacteria, fungus, parasite, or virus) in a fluid. Such a trapping efficiency may be for a single biological component or for a plurality of biological components (e.g., one or more bacteria and/or one or more fungi, and/or one or more parasite, and/or one or more virus) (e.g., trapped in a same relative location or different locations) in a given device. A device may have a separation efficiency of at least 50%, at least 60%, at least 70%, at least 80%, at least 90%, at least 95%, at least 97%, at least 99%, at least 99.5%, at least 99.9%, at least 99.9999%, at least 99.999999%, or 100% for a biological component (e.g., a bacteria, fungus). Such a device may separate a biological component from all others such that only the biological component is trapped in the device (e.g., and all others pass through or are lysed) or, for example, may trap the biological component separate from any other trapped biological component trapped in the device. A device may have a retrieval efficiency of at least 50%, at least 60%, at least 70%, at least 80%, at least 90%, at least 95%, at least 97%, at least 99%, at least 99.5%, at least 99.9%, at least 99.9999%, at least 99.999999%, or 100% for a biological component (e.g., a bacteria, fungus, parasite, or virus). Retrieval may occur by backflowing fluid (e.g., water, saline, media, solution, or buffer) through the device. A device may have a lysing efficiency of at least 50%, at least 60%, at least 70%, at least 80%, at least 90%, at least 95%, at least 97%, at least 99%, at least 99.5%, at least 99.9%, at least 99.9999%, at least 99.999999%, or 100% for a biological component (e.g., blood cells). A device may have a detection limit of at or below 5×10⁻10 M, at or below 5×10⁻¹² M, at or below 5×10⁻¹⁴ M, at or below 5×10⁻¹⁶ M, at or below 5×10⁻¹⁸ M, at or below 5×10⁻²⁰ M, or at or below 5×10⁻²² M for a biological component (e.g., a bacteria, fungus, parasite, or virus).

Methods, devices, and systems disclosed herein may include one or more components and/or one or more steps to alter rheology of a fluid, for example to improve flow through a device. Such components may be internal to a device or external to a device and disposed in a manner to act on the device (e.g., through an inlet) (e.g., at a distance). Heating a fluid (e.g., while it is in a device), mechanical energy (e.g., ultrasound), and one or more surfactants may be used to alter rheology, for example. Methods, devices, and systems disclosed herein may include one or more components and/or one or more steps to mitigate aggregates (e.g., agglomerates, coagulants). For example, anticoagulants may be used in a fluid to prevent coagulation of blood.

Methods, devices, and systems disclosed herein may be used to detect and/or identify infectious agents in a fluid. Such agents may be intracellular or extracellular. For example, a device may lyse cells thereby releasing any intracellular infectious agents that may then be trapped. Methods, devices, and systems disclosed herein may be used to detect and/or identify pathogens in a fluid. Such pathogens may be intracellular or extracellular. For example, a device may lyse cells thereby releasing any intracellular infectious agents that may then be trapped. Lysing and trapping may happen in a single unit (e.g., microfluidic unit) or in different units (e.g., microfluidic units) connected in serial in a device.

In certain embodiments, a device is operable to pass certain cells through channels (e.g., microfluidic channels). In some embodiments, such channels have a size and shape to allow one or more types of cells to pass through intact. In some embodiments, such channels have a size and shape to lyse one or more types of cells and pass through such cells by transporting lysate thereof. Channels may be arranged to selectively lyse cells. For example, channels may be arranged to lyse cells of one type and allow cells of another type to pass through. In general, larger channels are more likely to allow more types of cells (e.g., of different sizes) to pass through intact. Larger channels will also prevent passage. In this way, in some embodiments, a device may filter out large cells or other biological components that are undesirable (e.g., fungi, cancer cells, or contaminants) while maintaining integrity of other cells (e.g., blood cells).

In some embodiments, a device includes one or more second channels fluidically coupled to a first channel, for example in a biological component separation unit and/or a lysing unit. The one or more second channels may be significantly smaller than the first channel in order to promote lysing of one or more biological components (e.g., types of cells) and/or trapping of one or more biological components (e.g., types of cells). Trapping may occur because a biological component is too small to pass through the one or more second channels. Trapping may occur because a biological component is too small to enter into a first channel (and therefore gets trapped at an entrance to the first channel). Different components may be trapped in different locations. For example, large fungus cells may be trapped at an entrance to a first channel while other biological components (e.g., cells) may be free to travel into a first channel before being trapped. Lysing may occur at junction(s) between the first channel and the one or more second channels. Without wishing to be bound by any particular theory, lysing may occur due to a size difference between first and second channels and/or pressure applied (e.g., via suction) at one or more second channels.

The arrangement of channels (e.g., first and second channels) in a device may be used to facilitate separation of biological components based on mechanical properties (e.g., rigidity), for example even where components have a similar or same size. For example, where a first channel is fluidically coupled to a second channel, the channels may have different sizes and/or a relative orientation such that a relatively rigid biological component may not be able to distort (e.g., deform) to pass through the second channel (or enter the first channel) whereas a relatively soft and/or elastic biological component may be able to distort and pass through a second channel (e.g., intact or lysed). For example, cancer cells are generally more rigid than non-cancer cells (e.g., circulating tumor cells are more rigid than blood cells) and may not be able to so deform.

In some embodiments, a device is used to filter one or more biological components from a fluid. Fluid may be flowed continuously for a period of time such that channels (e.g., first channels) fill with the biological component. In some embodiments, fluid may continue to flow sufficiently (e.g., through second channels) to continue to filter biological component from the fluid. In some such embodiments, it may be desirable for a fluid to have low viscosity to promote flow as a biological component fills channels of a device. Thus, in some embodiments, it may be preferable to lyse cells in a fluid before flowing the fluid through a device, for example using a detergent. A detergent may be used to lyse at least 50%, at least 60%, at least 70%, at least 80%, at least 90%, at least 95%, or at least 99% of cells of one or more first types (e.g., blood cells) in a fluid while leaving cells of one or more second types (e.g., bacteria cells) intact (e.g., with no or reduced lysing) before flowing the fluid through a device to filter the cells of the one or more second types. Filtered biological component may then be retrieved, for example by backflow of fluid.

Biological components may be separated (e.g., isolated and/or filtered) and/or lysed by a device. Biological components may be trapped in and/or at channels in a device. Some biological components may pass through a device intact. A biological component may be cellular. A biological component may include cells. Cells of different sizes and/or mechanical properties may behave differently when interacting with channels in a device, for example one type of cells may be trapped at and/or in a channel and another type of cell may be lysed or pass through intact. A biological component may be a pathogen. A biological component may be an infectious agent. A biological component may be a bacteria, a eukaryotic cell (e.g., Plasmodium cells, yeast), a fungus (e.g., yeast), a prokaryotic cell, an exome, a virus (e.g., that is bound to a particle that may be trapped at and/or in a channel in a device), a virus-like particle, a vesicle (e.g., extracellular or intracellular vesicle). A biological component may be extracellular or intracellular. A device may lyse a first biological component to release and trap (e.g., simultaneously) a second biological component. A biological component, for example one that is lysed or passed through by a device, may be cellular, for example blood cells. A biological component may be blood cells, for example red blood cells and/or white blood cells.

It should be understood that the order of steps or order for performing certain action is immaterial so long as operability is not lost. Moreover, two or more steps or actions may be conducted simultaneously. As is understood by those skilled in the art, the terms “over”, “under”, “above”, “below”, “beneath”, and “on” are relative terms and can be interchanged as appropriate, for example in reference to different orientations of the layers, elements, and substrates included in the present disclosure. In some embodiments, a first layer on a second layer means a first layer directly on and in contact with a second layer. In some embodiments, a first layer on a second layer may include another layer therebetween.

U.S. Provisional Patent Application Ser. No. 63/737,557, filed Dec. 20, 2024, by Paulsson, et al., is incorporated herein by reference.

The following enumerated embodiments are expressly contemplated within the scope of the present disclosure. These enumerated embodiments are illustrative and are not intended to be limiting or exhaustive.
1. A device (e.g., microfluidic device) for separating one or more biological components [e.g., one or more infectious agents (e.g., one or more viruses, one or more bacteria, one or more fungi, one or more prokaryotic cell, one or more eukaryotic cell, or a combination thereof)] [e.g., one or more target cells (e.g., one or more non-blood cell, one or more mammalian cell, one or more human cell, one or more animal cell, one or more patient cell, one or more circulating tumor cell, one or more sickle cell, one or more rare cell, or a combination thereof)] in a fluid, the device comprising a plurality of biological component separation units fluidically connected in parallel, the units comprising channels (e.g., micro-and/or nano-channels) arranged (e.g., sized, shaped, and/or oriented) such that fluid flow through the units causes separation of biological components having different sizes and/or mechanical properties [e.g., by lysing one biological component and not another biological component (e.g., by trapping said another biological component)] [e.g., by trapping one biological component (e.g., at an entrance to a channel in the unit) and allowing another biological component to pass through and/or be lysed].
2. A device (e.g., microfluidic device) for lysing one or more biological components [e.g., cells (e.g., blood cells and/or epithelial cells)] in a fluid, the device comprising a plurality of biological component lysing units fluidically connected in parallel, the units comprising channels (e.g., micro-and/or nano-channels) arranged (e.g., sized, shaped, and/or oriented) such that fluid flow through the units causes lysing of one or more biological components (e.g., one or more target cells).
3. The device of embodiment 2, wherein the units comprise channels arranged (e.g., sized, shaped, and/or oriented) such that fluid flow through the units causes selective lysing of one or more biological components.
4. The device of any one of the preceding embodiments, wherein the plurality of units comprises at least 50,000 units, at least 100,000 units, at least 200,000, at least 300,000 units, at least 400,000 units, or at least 500,000 units, at least 1,000,000 units, at least 2,000,000 units, at least 3,000,000 units, at least 4,000,000 units, at least 5,000,000 units, at least 6,000,000 units, at least 7,000,000 units, at least 8,000,000 units, at least 9,000,000 units, at least 10,000,000 units, at least 20,000,000 units, at least 25,000,000 units, at least 50,000,000 units, at least 75,000,000 units, or at least 100,000,000 units connected in parallel.
5. The device of any one of the preceding embodiments, wherein the units are uniform in arrangement (e.g., are identically arranged as each other).
6. The device of any one of the preceding embodiments, wherein the units are uniform in size (e.g., wherein the channels comprised in each of the units are sized identically).
7. The device of any one of the preceding embodiments, wherein the units are arranged in a regular array (e.g., a 1D or 2D array).
8. The device of any one of the preceding embodiments, wherein the units comprise first channels (e.g., microchannels) and second channels (e.g., micro-and/or nano-channels), wherein the first channels and the second channels are arranged such that fluid flow through the device travels through the first channels then the second channels [e.g., and wherein the first channels are larger (e.g., in cross-sectional area (e.g., relative to a primary direction of fluid flow), height, width, or a combination thereof) than the second channels] (e.g., and the units are arranged such that the separation occurs at and/or in a first channel of the units) (e.g., and the units are arranged such that the lysing occurs at a junction of a first channel and one or more second channels of the units).
9. A device (e.g., microfluidic device) for separating and/or lysing one or more biological components in a fluid, the device comprising first channels (e.g., microchannels) (e.g., fluidically connected in parallel to each other) and second channels (e.g., micro-and/or nano-channels) (e.g., a subset of which is fluidically connected in parallel to each first channel) arranged such that fluid flow through the device travels through the first channels then the second channels, wherein the first channels are larger than the second channels.
10. The device of embodiment 8 or embodiment 9, wherein each of the first channels have at least one dimension that is larger than a corresponding at least one dimension of each of the second channels.
11. The device of any one of embodiments 8-10, wherein the at least one dimension is a cross section relative to a primary direction of fluid flow.
12. The device of any one of embodiments 8-11, wherein a ratio of the at least one dimension in the first channel to the corresponding at least one dimension in second channel is in a range of from 1 to 1000 (e.g., is at least 1.1, at least 1.2, at least 1.25, at least 1.3, at least 1.4, at least 1.5, at least 1.6, at least 1.7, at least 1.75, at least 1.8, at least 1.9, at least 2, at least 2.5, at least 3, at least 4, at least 5, at least 6, at least 7, at least 8, at least 9, at least 10, at least 20, at least 30, at least 40, at least 50, at least 60, at least 70, at least 80, at least 90, at least 100, at least 200, at least 300, at least 400, at least 500, at least 600, at least 70, at least 800, at least 900, or at least 1000).
13. The device of any one of embodiments 8-12, wherein the first channels and the second channels are non-parallel (e.g., to each other).
14. The device of any one of embodiments 8-13, wherein the second channels are arranged at an angle relative to the first channels in a range of from 0 to 180 degrees (e.g., from 5 to 175 degrees, from 10 to 170 degrees, from 15 to 165 degrees, from 20 to 160 degrees, from 25 to 155 degrees, from 30 to 150 degrees, from 35 to 145 degrees, from 40 to 140 degrees, from 45 to 135 degrees, from 50 to 130 degrees, from 55 to 125 degrees, from 60 to 120 degrees, from 65 to 115 degrees, from 70 to 110 degrees, from 75 to 105 degrees, from 80 to 100 degrees) (e.g., wherein the second channels are arranged perpendicular to the first channels).
15. The device of any one of embodiments 8-14, wherein each of the first channels is directly fluidically coupled (e.g., directly connected) to at least one of the second channels.
16. The device of embodiment 15, wherein the at least one of the second channels is a plurality of the second channels (e.g., at least 2, at least 3, at least 4, at least 5 of the second channels, and optionally no more than 10, no more than 9, no more than 8, no more than 7, no more than 6, or no more than 5 of the second channels) (e.g., connected in parallel to the first channel).
17. The device of embodiment 16, wherein the plurality of the second channels are mutually parallel.
18. The device of embodiment 16 or embodiment 17, wherein the plurality of the second channels are distributed (e.g., evenly) over a distance of no more than 100 μm, no more than 80 μm, no more than 60 μm, no more than 50 μm, no more than 40 μm, no more than 25 μm, no more than 20 μm, no more than 10 μm, no more than 9 μm, no more than 8 μm, no more than 7 μm, no more than 6 μm, no more than 5 μm, no more than 4 μm, no more than 3 μm, no more than 2 μm, or no more than 1 μm.
19. The device of any one of embodiments 16-18, wherein each adjacent pair of the plurality of the second channels is spaced apart by a distance of no more than 50 μm, no more than 40 μm, no more than 30 μm, no more than 20 μm, no more than 10 μm, no more than 8 μm, no more than 6 μm, no more than 5 μm, no more than 4 μm, no more than 3 μm, no more than 2 μm, no more than 1 μm, no more than 0.75 μm, no more than 0.5 μm, or no more than 0.25 μm.
20. The device of any one of embodiments 16-19, wherein, for each of the first channels, the plurality of the second channels are disposed (e.g., spaced and/or distributed) to apply pressure (e.g., a pressure gradient) at at least two distinct locations on a biological component corresponding in size to the first channel when the biological component is disposed in the first channel.
21. The device of any one of embodiments 15-20, wherein, for each of the first channels, the at least one of the second channels are disposed (e.g., spaced and/or distributed) relative to the first channel to apply a pressure gradient to a side of a biological component when disposed in the first channel.
22. The device of any one of embodiments 15-21, wherein, for each of the first channels, the at least one of the second channels are arranged at an angle relative to the first channel in a range of from 0 to 180 degrees (e.g., from 5 to 175 degrees, from 10 to 170 degrees, from 15 to 165 degrees, from 20 to 160 degrees, from 25 to 155 degrees, from 30 to 150 degrees, from 35 to 145 degrees, from 40 to 140 degrees, from 45 to 135 degrees, from 50 to 130 degrees, from 55 to 125 degrees, from 60 to 120 degrees, from 65 to 115 degrees, from 70 to 110 degrees, from 75 to 105 degrees, from 80 to 100 degrees) (e.g., wherein the second channels are arranged perpendicular to the first channels).
23. The device of any one of embodiments 8-22, wherein the first channels have a width and/or a height in a range of from 50 nm to 15 μm (e.g., from 1 μm to 10 μm).
24. The device of any one of embodiments 8-23, wherein the first channels have a length of at least 10 μm (e.g., at least 20 μm, at least 30 μm, at least 40 μm, or at least 50 μm).
25. The device of any one of embodiments 8-24, wherein the second channels have a width and/or a height in a range of from 20 nm to 800 nm.
26. The device of any one of embodiments 8-25, wherein the second channels have a length of at least 500 nm (e.g., at least 1 μm, at least 2 μm, at least 3 μm, or at least 4 μm).
27. The device of any one of embodiments 8-26, comprising third microchannels (e.g., wherein the units comprise the third microchannels) larger than the second channels, wherein fluid flow through the device travels through the second channels then the third microchannels.
28. The device of embodiment 27, wherein the third channels are disposed parallel to the first channels.
29. The device of embodiment 27 or embodiment 28, wherein the third channels have a same size as the first channels.
30. The device of any one of embodiments 27-29, wherein the third channels are directly fluidically coupled to a common fluid collection channel.
31. The device of any one of embodiments 8-30, wherein the first channels and/or the second channels are linear.
32. The device of any one of embodiments 8-31, wherein the first channels and/or the second channels are non-linear (e.g., are curvilinear).
33. The device of any one of embodiments 8-32, comprising a common fluid distribution channel, wherein each of the first channels is directly fluidically coupled to the common fluid distribution channel.
34. The device of any one of embodiments 8-33, comprising an inlet and an outlet, wherein the first channels and the second channels are arranged such that all flow from the inlet to the outlet travels through the first channels and then the second channels.
35. The device of any one of embodiments 8-34, wherein the first channels and the second channels are sized and arranged such that at least 70% (e.g., at least 80%, at least 90%, at least 95%, at least 99%) (e.g., all) bacteria in fluid that is flowed through the first channels and the second channels are separated [e.g., are removably trapped at or in the device (e.g., at or in the first channels or the second channels) (e.g., at or in the first channels)].
36. The device of any one of embodiments 8-35, wherein, for each of the first channels, a proximal end of the first channel comprises a flared opening (e.g., a curved flared opening).
37. The device of any one of embodiments 8-36, wherein the first channels have a size corresponding to a biological component [e.g., one or more infectious agents (e.g., one or more viruses, one or more bacteria, one or more fungi, one or more prokaryotic cell, one or more eukaryotic cell, or a combination thereof)] or target cell (e.g. one or more non-blood cell, one or more mammalian cell, one or more human cell, one or more animal cell, one or more patient cell, one or more circulating tumor cell, one or more sickle cell, one or more rare cell, or a combination thereof).
38. The device of any one of embodiments 8-37, wherein the second channels have a size smaller than a (e.g., the) biological component.
39. The device of any one of embodiments 9-38, comprising biological component separation units comprising ones of the first channels and ones of the second channels (e.g., and ones of the third channels) (e.g., each comprising one of the first channels, one or more of the second channels, and, if present, one of the third channels).
40. The device of any one of embodiments 9-38, comprising biological component lysing units comprising ones of the first channels and the second channels (e.g., and the third channels) (e.g., each comprising one of the first channels, one or more of the second channels, and, if present, one of the third channels).
41. The device of embodiment 39 or embodiment 40, wherein the units comprise channels arranged (e.g., sized, shaped, and/or oriented) such that fluid flow through the units causes selective lysing of one or more biological components.
42. The device of any one of embodiments 39-41, wherein the plurality of units comprises at least 50,000 units, at least 100,000 units, at least 200,000, at least 300,000 units, at least 400,000 units, or at least 500,000 units, at least 1,000,000 units, at least 2,000,000 units, at least 3,000,000 units, at least 4,000,000 units, at least 5,000,000 units, at least 6,000,000 units, at least 7,000,000 units, at least 8,000,000 units, at least 9,000,000 units, at least 10,000,000 units, at least 20,000,000 units, at least 25,000,000 units, at least 50,000,000 units, at least 75,000,000 units, or at least 100,000,000 units connected in parallel.
43. The device of any one of embodiments 39-42, wherein the units are uniform in arrangement (e.g., are identically arranged as each other).
44. The device of any one of embodiments 39-43, wherein the units are uniform in size (e.g., wherein the channels comprised in each of the units are sized identically).
45. The device of any one of embodiments 39-44, wherein the units are arranged in a regular array (e.g., a 1D or 2D array).
46. The device of any one of embodiments 8-45, comprising the fluid disposed in the first channels and the second channels (e.g., and the third channels).
47. The device of embodiment 46, wherein the fluid comprises one or more detergents (e.g., a saponin).
48. The device of any one of embodiments 8-47, wherein the first channels and the second channels are disposed in a patterned layer (e.g., an elastomer layer) (e.g., a microfluidic layer) [e.g., bounded by plastic or glass (e.g., a coverslip)].
49. The device of any one of embodiments 8-48, wherein the first channels and the second channels are arranged (e.g., sized, shaped, and/or oriented) to trap (e.g., removably trap) one or more infectious agents (e.g., one or more viruses, one or more bacteria, one or more fungi, one or more prokaryotic cell, one or more eukaryotic cell, or a combination thereof) or target cell (e.g. one or more non-blood cell, one or more mammalian cell, one or more human cell, one or more animal cell, one or more patient cell, one or more circulating tumor cell, one or more sickle cell, one or more rare cell, or a combination thereof) in and/or at the first channels.
50. The device of any one of embodiments 8-49, wherein the first channels and the second channels are arranged (e.g., sized, shaped, and/or oriented) to lyse one or more cellular biological components (e.g., blood cells and/or epithelial cells) using (e.g., with) the first channels and the second channels (e.g., at junctions of one or more second channels with a first channel).
51. The device of any one of the preceding embodiments, wherein the device is a microfluidic device.
52. The device of any one of the preceding embodiments, wherein the device is operable to separate (e.g., isolate) one or more biological components in a fluid (e.g., blood, urine, saliva, sputum, tissue) [e.g., from one or more other biological components (e.g., cells) in the fluid].
53. The device of any one of embodiments 1-52, wherein the device is operable to selectively lyse one or more biological components [e.g., cells (e.g., blood cells)] in a fluid (e.g., blood, urine, saliva, sputum).
54. The device of embodiment 53, wherein the device is operable to selectively lyse the one or more biological components while preserving one or more other biological components [e.g., one or more infectious agents (e.g., one or more viruses, one or more bacteria, one or more fungi, one or more prokaryotic cell, one or more eukaryotic cell, one or more mammalian cell, one or more human cell, one or more animal cell, one or more patient cell, or a combination thereof)] in the fluid.
55. The device of any one of the preceding embodiments, wherein the one or more biological components (e.g., the biological component) comprises one or more infectious agents (e.g., one or more viruses, one or more bacteria, one or more fungi, one or more prokaryotic cell, one or more eukaryotic cell, or a combination thereof) or comprises one or more target cells (e.g. one or more non-blood cell, one or more mammalian cell, one or more human cell, one or more animal cell, one or more patient cell, one or more circulating tumor cell, one or more sickle cell, one or more rare cell, or a combination thereof).
56. The device of any one of the preceding embodiments, wherein the one or more biological components (e.g., the biological component) comprises one or more cellular components.
57. The device of any one of the preceding embodiments, wherein the fluid is or is derived from blood (e.g., whole or dilute), urine, stool, lymph, tissue, nasal or cheek swab, mucus, saliva, sputum, cerebrospinal fluid, breast milk, fluid aspirate, or wound or abscess drainage.
58. The device of any one of embodiments 1-56, wherein the fluid is waste water or environmental droplets.
59. The device of any one of embodiments 1-56, wherein the fluid is a pharmaceutical composition (e.g., a fluid comprising a drug or biologic).
60. The device of any one of embodiments 1-56, wherein the fluid is or is derived from food, drinking water, juice, milk, wine, or a beverage.
61. The device of any one of the preceding embodiments, wherein the channels (e.g., the first channels and the second channels) are visible through the device.
62. The device of any one of the preceding embodiments, wherein the device is transparent [e.g., is compatible with performing a characterization technique (e.g., brightfield, quantitative phase microscopy, hyperspectral imaging, instant structured illumination microscopy, Raman spectroscopy) on one or more biological components disposed in the device].
63. The device of any one of embodiments 1-8, wherein the units are arranged in a random arrangement.
64. The device of any one of embodiments 1-8, wherein the units are arranged in a radial arrangement.
65. A method comprising, in a microfluidic device, mechanically lysing cells using micro and/or nano-channels (e.g., first channels) thereby releasing virus, binding the virus to particles (e.g., in a reservoir of a device disposed in a fluid pathway after a first set of micro-and/or nano-channels), and trapping the particles with the micro-and/or nano-channels.
66. The method of embodiment 65, comprising retrieving the particles from the device and characterizing the virus.
67. A device comprising:
   first biological component separation units connected in parallel, wherein the biological component separation units are arranged (e.g., sized, shaped, and/or oriented) to trap a first biological component in a fluid; and
   second biological component separation units connected in parallel, wherein the first biological component separation units are fluidically connected in series with the second biological component separation units.
68. The device of embodiment 67, wherein the first biological component separation units and/or the second biological component separation units are also lysing units.
69. A method of separating (e.g., isolating) one or more biological components [e.g., one or more infectious agents (e.g., one or more viruses, one or more bacteria, one or more fungi, one or more prokaryotic cells, one or more eukaryotic cells, or a combination thereof)] (e.g., cells) in a fluid (e.g., blood, urine, lymphatic fluid, saliva, sputum) and/or purifying a fluid, the method comprising flowing a fluid comprising a first biological component through channels (e.g., microchannels) in a device (e.g., a microfluidic device) such that at least 50% of the first biological component in the fluid that flows through the channels is trapped in and/or at the channels (e.g., one or more entrances thereto).
70. The method of embodiment 69, wherein the at least 50% of the first biological component in the fluid that is trapped is removably trapped.
71. The method of embodiment 69 or embodiment 70, wherein at least 60%, at least 70%, at least 80%, at least 90%, at least 95%, at least 98%, at least 99%, or at least 99.5% of the first component is trapped by flowing the fluid.
72. The method of any one of embodiments 69-71, comprising removing at least a portion (e.g., at least 50%, at least 60%, at least 70%, at least 80%, at least 90%, at least 95%, or at least 99%) of the at least 50% of the first biological component in the fluid from the channels (e.g., by reversing a direction of fluid flow).
73. The method of any one of embodiments 69-72, wherein the fluid comprises a second biological component and no more than 10% (e.g., no more than 5%, no more than 3%, no more than 2%, no more than 1%, no more than 0.5%, no more than 0.1%, no more than 0.01%, or no more than 0.001%) of the second biological component in the fluid that flows through the channels is trapped (e.g., isolated) in the device.
74. The method of any one of embodiments 69-73, wherein the fluid comprises a second biological component (e.g., cells of a different type from the first biological component) and the second component is not trapped during flowing of the fluid.
75. The method of embodiment 73 or embodiment 74, wherein the second biological component is lysed using the channels.
76. The method of any one of embodiments 73-75, wherein flowing the fluid causes the second biological component to be lysed in the device.
77. The method of any one of embodiments 69-75, wherein the method is performed at a fluid flow rate of at least 1 μL/min, (e.g., at least 5 μm/min, at least 10 μm/min, at least 25 μL/min, at least 50 μL/min, at least 75 μL/min, at least 0.1 mL/min, at least 0.25 mL/min, at least 0.5 mL/min, at least 0.75 mL/min, at least 1 mL/min, at least 5 mL/min, at least 10 mL/min, at least 20 mL/min, at least 25 mL/min, at least 50 mL/min, at least 100 mL/min, or at least 200 mL/min).
78. The method of any one of embodiments 69-77, comprising providing the fluid [e.g., from a reservoir (e.g., syringe, vacutainer tube, conical tube, or blood culture bottle)] to the device (e.g., at an inlet thereof) thereby causing the fluid to flow through the device.
79. The method of any one of embodiments 69-78, comprising providing a detergent (e.g., a saponin) in the fluid.
80. The method of embodiment 79, wherein the detergent is provided before the fluid is provided to the device (e.g., by mixing in a syringe or conical tube (e.g., immediately) prior to providing the fluid).
81. The method of embodiment 79, wherein the detergent is provided to the fluid in the device.
82. The method of any one of embodiments 69-81, wherein the method is performed for a volume of fluid of at least 0.1 mL (e.g., at least 0.2 mL, at least 0.5 mL, at least 0.75 mL, at least 1 mL, at least 2 mL, at least 10 mL, at least 25 mL, at least 50 mL, at least 75 mL, at least 100 mL, at least 200 mL, at least 500 mL, at least 750 mL, at least 1 L, at least 2 L, at least 3 L, at least 4 L, at least 5 L, at least 10 L), preferably over a period of no more than 2 h, no more than 1 h, no more than 30 min, no more than 20 min, no more than 10 min, no more than 5 min, no more than 2 min, or no more than 1 min).
83. The method of any one of embodiments 69-82, comprising determining an identity of the first biological component while the first biological component is trapped in the device.
84. The method of any one of embodiments 69-83, comprising imaging the first biological component after trapping.
85. The method of any one of embodiments 69-84, comprising imaging the first biological component while trapped on the device.
86. The method of any one of embodiments 69-85, comprising retrieving the first biological component from the device (e.g., by backflowing fluid through the device).
87. The method of embodiment 86, comprising analyzing (e.g., genotyping or phenotyping) the first biological component after retrieval.
88. The method of any one of embodiments 69-87, wherein the fluid is derived from a subject and the method comprises determining whether the subject has an infection (e.g., from a parasite), a disease, cancer, or a genetic disorder based on an identity of the first biological component.
89. A method of separating (e.g., isolating) one or more biological components [e.g., one or more infectious agents (e.g., one or more viruses, one or more bacteria, one or more fungi, or a combination thereof)] in a fluid (e.g., blood, urine, lymph, saliva, sputum) lysing a biological component in a fluid, and/or purifying a fluid, the method comprising:
   flowing a fluid comprising a cellular biological component (e.g., through a device); and
   applying (e.g., via suction) one or more pressures (e.g., one or more pressure gradients) to the biological component with the channels while the fluid is flowing, thereby lysing the biological component.
90. The method of embodiment 89, wherein the biological component comprises units (e.g., cells) [e.g., each having an outer perimeter (e.g., a cell wall or cell membrane)], the one or more pressures is a plurality of pressures, wherein the pressures are applied to multiple discrete locations on each of the units (e.g., on the outer perimeter).
91. The method of embodiment 89 or embodiment 90, wherein the one or more pressures are applied to a weak portion (e.g., weakened by detergent and/or a weak side) of the biological component.
92. The method of any one of embodiments 89-91, wherein the fluid is flowed through a device comprising channels (e.g., microchannels) and the one or more pressures is applied using the channels.
93. The method of any one of embodiments 89-92, wherein the one or more pressures is applied using micro-and/or nano-channels (e.g., wherein the channels comprise micro-and/or nano-channels).
94. The method of any one of embodiments 89-93, wherein application of the one or more pressures result in lysing of at least 60% of the biological component in the fluid that is flowed (e.g., at least 70%, at least 80%, at least 90%, at least 95%, at least 99%, at least 99.5%, at least 99.9%, at least 99.99%, at least 99.9999% or at least 99.999999%).
95. The method of any one of embodiments 89-94, wherein the biological component is a first biological component and the fluid comprises a second biological component that is not lysed by the channels.
96. The method of any one of embodiments 89-95, wherein the biological component is a first biological component and the fluid comprises a second biological component at least a portion (e.g., at least 50%, at least 60%, at least 70%, at least 80%, at least 90%, at least 95%, at least 97%, at least 99%) of which is trapped by the channels and/or passes through the channels.
97. The method of any one of embodiments 89-96, wherein the first biological component has a second biological component disposed therein and the method comprises trapping the second biological component with the channels.
98. A method of (e.g., selectively) lysing cells (e.g., blood cells), the method comprising mechanically lysing cells in a fluid by flowing the fluid through channels in a device.
99. The method of embodiment 98, wherein the cells that are lysed are first cells and the method comprises separating [e.g., isolating and/or trapping (e.g., individually] second cells using the channels.
100. The method of embodiment 99, wherein the lysing occurs in a first stage of the device (e.g., using a first subset of the channels) and the separating occurs in a second stage of the device (e.g., a second subset of the channels).
101. The method of any one of embodiments 98-100, wherein the cells have a second biological component disposed therein and the method comprises trapping the second biological component with the channels.
102. The method of any one of embodiments 98-101, wherein the channels are micro-and/or nano-channels.
103. The method of any one of embodiments 98-102, wherein a lysing efficiency is at least 50% (e.g., at least 60%, at least 70%, at least 80%, at least 90%, at least 95%, at least 97%, at least 99%, at least 99.5%, or at least 99.9%).
104. An article, comprising:
   a microfluidic device defining a plurality of repeat units, each of the repeat units comprising a first microfluidic channel, and at least 3 second channels (e.g. nanofluidic channels) each fluidically connecting to the first microfluidic channel,
   wherein the first microfluidic channel has a maximum cross-sectional dimension less than 5 micrometers, and
   wherein the second channels have a maximum cross-sectional dimension less than 0.5 micrometers.
105. The article of embodiment 104, wherein the second channels are substantially parallel.
106. The article of any one of embodiments 104-105, wherein the microfluidic device defines at least 1,000 repeat units.
107. The article of any one of embodiments 104-106, wherein the microfluidic device defines at least 3,000 repeat units.
108. The article of any one of embodiments 104-107, wherein the microfluidic device defines at least 10,000 repeat units.
109. The article of any one of embodiments 104-108, wherein the microfluidic device defines at least 30,000 repeat units.
110. The article of any one of embodiments 104-109, wherein the microfluidic device defines at least 100,000 repeat units, at least 1,000,000 repeat units, at least 2,000,000 repeat units, at least 3,000,000 repeat units, at least 4,000,000 repeat units, at least 5,000,000 repeat units, at least 6,000,000 repeat units, at least 7,000,000 repeat units, at least 8,000,000 repeat units, at least 9,000,000 repeat units, at least 10,000,000 repeat units, at least 20,000,000 repeat units, at least 25,000,000 repeat units, at least 50,000,000 repeat units, at least 75,000,000 repeat units, or at least 100,000,000 repeat units.
111. The article of any one of embodiments 104-110, wherein the repeat units are all identical.
112. The article of any one of embodiments 104-111, wherein the substantially parallel second channels are substantially identical.
113. The article of any one of embodiments 104-112, wherein the microfluidic device comprises at least 5 substantially parallel channels each fluidically connecting the first microfluidic channel to the second microfluidic channel.
114. The article of any one of embodiments 104-113, wherein the microfluidic device further comprises an inlet and an outlet in fluid communication with the plurality of repeat units.
115. The article of embodiment 114, wherein each flow path through the microfluidic device between the inlet and the outlet passes through a repeat unit of the plurality of repeat units.
116. The article of any one of embodiments 114 or 115, and wherein each flow path through the microfluidic device between the inlet and the outlet passes through only one repeat unit of the plurality of repeat units.
117. The article of any one of embodiments 114-116, wherein the microfluidic device further defines one or more flow channels in fluid communication with the inlet, wherein the one or more flow channels is in fluid communication with the plurality of repeat units.
118. The article of any one of embodiments 114-117, wherein the microfluidic device further defines one or more waste channels in fluid communication with the outlet, wherein the one or more waste channels is in fluid communication with the plurality of repeat units.
119. The article of any one of embodiments 104-118, wherein the first microfluidic channel of the repeat units has a flared entrance.
120. The article of any one of embodiments 104-119, wherein the microfluidic device comprises glass, and the repeat units are defined within the glass.
121. The article of any one of embodiments 104-120, wherein the microfluidic device comprises a polymer, and the repeat units are defined within the polymer.
122. The article of any one of embodiments 104-121, wherein the article is a removable cartridge.
123. The article of any one of embodiments 104-122, wherein a ratio of a length of the first microfluidic channel to a length of the second channels is between 0.001 and 1000.
124. The article of any one of embodiments 104-123, wherein a ratio of a cross-sectional dimension of the first microfluidic channel to a cross-sectional dimension of the second channels is between 0.001 and 1000.
125. The article of any one of embodiments 104-124, wherein a ratio of a cross-sectional area of the first microfluidic channel to a cross-sectional area of the second channels is between 0.001 and 1000.
126. The article of any one of embodiments 104-125, wherein a ratio of a volume of the first microfluidic channel to a volume of the second channels is between 0.001 and 1000.
127. The article of any one of embodiments 104-126, wherein the article further comprises a collection channel substantially parallel to the first microfluidic channel, and the second channels each fluidically connect to the collection channel
128. The article of embodiment 127, wherein a ratio of a length of the first microfluidic channel to a length of the collection channel is between 0.001 and 1000.
129. The article of any one of embodiments 127 or 128, wherein a ratio of a cross-sectional dimension of the first microfluidic channel to a cross-sectional dimension of the collection channel is between 0.001 and 1000.
130. The article of any one of embodiments 127-129, wherein a ratio of a cross-sectional area of the first microfluidic channel to a cross-sectional area of the collection channel is between 0.001 and 1000.
131. The article of any one of embodiments 127-130, wherein a ratio of a volume of the first microfluidic channel to a volume of the collection channel is between 0.001 and 1000.
132. The article of any one of embodiments 104-131, wherein the first microfluidic channel is substantially linear.
133. The article of any one of embodiments 104-132, wherein the first microfluidic channel is curvilinear.
134. The article of any one of embodiments 104-133, wherein the first microfluidic channel is non-linear.
135. The article of any one of embodiments 104-134, wherein at least some of the second channels are substantially linear.
136. The article of any one of embodiments 104-135, wherein at least some of the second channels are curvilinear.
137. The article of any one of embodiments 104-136, wherein at least some of the second channels are non-linear.
138. The article of any one of embodiments 104-137, wherein the first microfluidic channel is substantially linear.
139. The article of any one of embodiments 104-138, wherein the first microfluidic channel is curvilinear.
140. The article of any one of embodiments 104-139, wherein the first microfluidic channel is non-linear.
141. The article of any one of embodiments 104-140, wherein at least some of the second channels are substantially linear.
142. The article of any one of embodiments 104-141, wherein at least some of the second channels are curvilinear.
143. The article of any one of embodiments 104-142, wherein at least some of the second channels are non-linear.
144. An article, comprising:
   a microfluidic device defining a plurality of repeat units, each of the repeat units comprising a first microfluidic channel, a collection microfluidic channel substantially parallel to the first microfluidic channel, and at least 3 substantially parallel second channels (e.g., nanochannels) each fluidically connecting the first microfluidic channel to the collection microfluidic channel,
   wherein the first microfluidic channel has a maximum cross-sectional dimension less than 5 micrometers, and
   wherein the second channels have a maximum cross-sectional dimension less than 0.5 micrometers.
145. A method, comprising:
   providing a fluid containing a first type of cells and second entities, wherein the first type of cells has an average volume bigger than the second entities;
   distorting the first type of cells to have an aspect ratio of at least 2:1; and
   flowing the fluid through a microfluidic channel such that the first type of cells pass therethrough and the second entities do not pass therethrough.
146. The method of embodiment 145, wherein the first type of cells are mammalian cells.
147. The method of any one of embodiments 145 or 146, wherein the first type of cells comprise tumor cells.
148. The method of any one of embodiments 145-147, wherein the first type of cells comprise circulating tumor cells.
149. The method of any one of embodiments 145-148, wherein the first type of cells comprise bacterial cells.
150. The method of any one of embodiments 145-149, wherein the first type of cells comprise parasitic cells.
151. The method of any one of embodiments 145-150, wherein the first type of cells comprise fungal cells.
152. The method of any one of embodiments 145-151, further comprising lysing the first type of cells to cause the first type of cells to pass through the microfluidic channel.
153. The method of any one of embodiments 145-152, wherein the fluid comprises blood.
154. The method of any one of embodiments 145-153, wherein the fluid comprises red blood cells.
155. The method of any one of embodiments 145-154, wherein the fluid comprises sickle red blood cells.
156. The method of any one of embodiments 145-155, wherein the fluid comprises white blood cells.
157. The method of any one of embodiments 145-156, wherein the fluid comprises untreated blood.
158. The method of any one of embodiments 145-157, wherein the fluid comprises urine.
159. The method of any one of embodiments 145-158, wherein the fluid comprises sputum.
160. The method of any one of embodiments 145-159, wherein the fluid comprises mucus.
161. The method of any one of embodiments 145-160, wherein the fluid comprises cerebrospinal fluid.
162. The method of any one of embodiments 145-161, wherein the fluid comprises lymph.
163. The method of any one of embodiments 145-162, wherein the fluid comprises stool.
164. The method of any one of embodiments 145-163, wherein the fluid comprises tissue.
165. The method of any one of embodiments 145-164, wherein the fluid comprises breast milk.
166. The method of any one of embodiments 145-165, wherein the fluid comprises fluid aspirate.
167. The method of any one of embodiments 145-166, wherein the fluid comprises wound drainage.
168. The method of any one of embodiments 145-167, wherein the fluid comprises abscess.
169. The method of any one of embodiments 145-168, wherein the fluid comprises food.
170. The method of any one of embodiments 145-169, wherein the fluid comprises juice.
171. The method of any one of embodiments 145-170, wherein the fluid comprises wastewater.
172. The method of any one of embodiments 145-171, wherein the fluid comprises saline.
173. The method of any one of embodiments 145-172, wherein the fluid is aqueous.
174. The method of any one of embodiments 145-173, wherein the fluid further comprises a surfactant.
175. The method of embodiment 174, wherein the surfactant alters an elasticity of the first type of cells.
176. The method of any one of embodiments 145-175, wherein the second entities comprise a second type of cell.
177. The method of any one of embodiments 145-176, wherein the second entities comprise particles.
178. The method of any one of embodiments 145-177, wherein the second entities comprise bacteria.
179. The method of any one of embodiments 145-178, wherein the second entities comprise parasitic cells.
180. The method of any one of embodiments 145-179, wherein the second entities comprise Plasmodium cells.
181. The method of any one of embodiments 145-180, wherein the second entities comprise exosomes.
182. The method of any one of embodiments 145-181, wherein the second entities comprise viruses or virus-like particles.
183. The method of any one of embodiments 145-182, wherein the second entities comprise extracellular vesicles or intracellular vesicles.
184. The method of any one of embodiments 145-183, wherein the second entities comprise fungi.
185. The method of any one of embodiments 145-184, wherein the second entities comprise yeast.
186. The method of any one of embodiments 145-185, further comprising determining the second entities.
187. The method of embodiment 186, comprising determining the second entities within the microfluidic channel.
188. The method of any one of embodiments 186 or 187, further comprising removing the second entities from the microfluidic channel, and determining the second entities after removal from the microfluidic channel.
189. The method of any one of embodiments 186-188, wherein determining the second entities comprises determining an identity of the second entities.
190. The method of any one of embodiments 186-189, wherein determining the second entities comprises sequencing the second entities.
191. The method of any one of embodiments 186-190, wherein determining the second entities comprises determining the second entities using FISH.
192. The method of any one of embodiments 186-191, wherein determining the second entities comprises determining the second entities using PCR.
193. The method of any one of embodiments 186-191, wherein determining the second entities comprises applying multi-omic analysis to the second entities.
194. The method of any one of embodiments 145-192, wherein flowing the fluid through the microfluidic channel comprises flowing the fluid using a syringe.
195. The method of any one of embodiments 145-193, wherein the fluid arises from a subject.
196. The method of embodiment 195, wherein the subject is human.
197. The method of embodiment 195, wherein the fluid is an extract from a plant or animal.
198. The method of any one of embodiments 195-197, wherein the method is a method of determining cancer in the subject.
199. The method of any one of embodiments 195-198, wherein the method is a method of determining a disease in the subject.
200. The method of any one of embodiments 195-199, wherein the method is a method of determining a genetic disorder in the subject.
201. The method of any one of embodiments 195-200, wherein the method is a method of determining a parasite in the subject.
202. The method of any one of embodiments 195-201, wherein the method is a method of determining an infection (e.g. bacteremia or sepsis) in the subject.
203. The method of any one of embodiments 195-202, wherein the infection is a bacterial infection.
204. The method of any one of embodiments 195-203, wherein the infection is a viral infection.
205. A method, comprising:
   providing a fluid containing a first type of cells and second entities, wherein the first type of cells has an average volume bigger than the second entities;
   distorting the first type of cells to have an aspect ratio of at least 2:1; and
   applying a force to the distorted first type of cells to cause the first type of cells to pass through a microfluidic channel dimensioned to prevent the second entities from flowing therethrough.
206. A method, comprising:
   providing a fluid containing a first type of cells and a second entities, wherein the first type of cells has an average volume bigger than the second entities;
   flowing the fluid through a first microfluidic channel having a volume less than 120% of the first type of cells and a maximum cross-sectional dimension less than 60% of the average diameter of a perfect sphere having the same volume as the first type of cells;
   flowing the first type of cells through a second microfluidic channel intersecting with the first microfluidic channel without flowing the second entities through the second microfluidic channel.
207. a method, comprising:
   flowing a fluid containing a first type of cells and a second entities through a microfluidic channel having a cross-sectional diameter smaller than a diameter of a perfect sphere having the same volume as the type of cells having the smaller volume, such that the first type of cells pass therethrough and the second entities do not pass therethrough.
208. The method of embodiment 207, further comprising collecting the second entities from the microfluidic channel.
209. The method of any one of embodiments 207 or 208, comprising distorting the first type of cells to cause the first type of cells to pass through the microfluidic channel.
210. The method of embodiment 209, comprising distorting the first type of cells to have an aspect ratio of at least 2:1.
211. The method of any one of embodiments 207-210, comprising lysing the first type of cells to cause the first type of cells to pass through the microfluidic channel.
212. A method, comprising:
   flowing a fluid containing blood cells and bacteria through a microfluidic filter such that at least 90% of the bacteria are trapped by the microfluidic filter and at least 90% of the blood cells pass through the microfluidic filter; and
   backflushing a second fluid through the microfluidic filter to recover at least 90% of the bacteria trapped by the microfluidic filter.
213. A method, comprising:
   providing a fluid containing a first type of cells and second entities, wherein the first type of cells has an average volume bigger than the second entities; and
   flowing the fluid through a microfluidic filter such that at least 90% of the second entities are trapped by the microfluidic filter and at least 90% of the first type of cells pass through the microfluidic filter.
214. A method, comprising:
   providing a fluid containing a first type of cells and second entities, wherein the first type of cells has an average volume bigger than the second entities; and
   flowing the fluid through a microfluidic filter such that at least 90% of the second entities are trapped by the microfluidic filter and at least 90% of the first type of cells are lysed by the microfluidic filter.
215. A method, comprising:
   providing a fluid containing cells and particles, wherein cells have an average volume bigger than the particles; and
   flowing the fluid through a microfluidic filter such that at least 90% of the particles are trapped by the microfluidic filter and at least 90% of the cells pass through the microfluidic filter.
216. A method, comprising:
   providing a fluid containing cells and particles, wherein cells have an average volume bigger than the particles; and
   flowing the fluid through a microfluidic filter such that at least 90% of the particles are trapped by the microfluidic filter and at least 90% of the cells are lysed by the microfluidic filter.
217. A method of determining cells; comprising:
   passing a fluid comprising a first type of cells suspected of containing therein a second type of cells through a microfluidic filter such that the first type of cells are lysed and the second type of cells are trapped by the microfluidic filter; and
   determining a distribution of the second type of cells within the microfluidic filter to determine containment of the second type of cells within the first type of cells.
218. The method of embodiment 217, wherein the method of determining cells is a method of determining a clinical diagnosis.
219. The method of any one of embodiments 217-218, wherein the method of determining clinical diagnosis can determine salmonella.
220. The method of any one of embodiments 217-219, wherein the method of determining clinical diagnosis can determine malaria.
221. The method of any one of embodiments 217-220, wherein the first type of cells is infected by the second type of cells.
222. The method of any one of embodiments 217-221, wherein the second type of cells are suspected of causing sepsis.
223. The method of any one of embodiments 217-222, wherein the fluid comprises blood.
224. A method of determining a viral infection; comprising:
   passing a fluid comprising cells suspected of being infected by a virus through a microfluidic filter such that the cells are lysed and the viruses are trapped by the microfluidic filter; and
   determining a distribution of the viruses within the microfluidic filter to determine infection of the cells by the virus.
225. A method, comprising:
   distorting a mammalian cell in a first direction such that it has an aspect ratio of at least 2:1;
   distorting the mammalian cell in a second direction different from the first direction; and
   flowing the distorted mammalian cell through a microfluidic channel.
226. The method of embodiment 225, wherein the first direction and the second direction define an angle of at least 30°.
227. The method of any one of embodiments 225 or 226, wherein the first direction and the second direction define an angle between 0° and 180°.
228. The method of any one of embodiments 225-227, wherein the second direction is orthogonal to the first direction.
229. The method of any one of embodiments 225-228, wherein the mammalian cell is a blood cell.
230. The method of any one of embodiments 225-228, wherein the mammalian cell is a red blood cell.
231. The method of any one of embodiments 225-228, wherein the mammalian cell is a white blood cell.
232. The method of any one of embodiments 225-231, comprising distorting the mammalian cell such that it has an aspect ratio of at least 4:1.
233. The method of any one of embodiments 225-232, wherein distorting the mammalian cell in a first direction comprises confining the mammalian cell in a confining microfluidic channel.
234. The method of embodiment 233, wherein the confining microfluidic channel has a volume less than 120% of the cell.
235. The method of any one of embodiments 233 or 234, wherein the confining microfluidic channel has a maximum cross-sectional dimension less than 60% of the diameter of a perfect sphere having the same volume as the mammalian cell.
236. The method of any one of embodiments 233-235, wherein the confining microfluidic channel has a maximum cross-sectional dimension less than 5 micrometers.
237. The method of any one of embodiments 225-236, wherein the mammalian cell is exposed to a surfactant.
238. The method of embodiment 237, wherein the surfactant alters an elasticity of the mammalian cell.
239. A method, comprising:
   distorting a mammalian cell in a first direction such that it has an aspect ratio of at least 2:1; and
applying a force to the mammalian cell in a second direction different from the first direction sufficient to move the mammalian cell in the second direction.
240. The method of embodiment 239, wherein the force applied to the mammalian cell in the second direction is sufficient to lyse the mammalian cell.
241. The method of any one of embodiments 239 or 240, comprising applying a force to a stretched portion of the mammalian cell.
242. The method of any one of embodiments 239-241, wherein the first direction is defined by a first microfluidic channel, and the second direction is defined by a second microfluidic channel intersecting the first microfluidic channel.
243. The method of embodiment 242, wherein a fluid flows from the first microfluidic channel to the second microfluidic channel.
244. The method of embodiment 243, wherein the fluid in the first microfluidic channel has a first Reynolds Number, and the fluid in the second microfluidic channel has a second Reynolds Number different from the first Reynolds Number.
245. A method of lysing a cell, comprising:
   distorting a mammalian cell such that it has an aspect ratio of at least 2:1; and
   applying a force to the distorted mammalian cell sufficient to lyse the cell.
246. The method of embodiment 245, comprising applying the force to a stretched portion of the distorted mammalian cell.
247. The method of any one of embodiments 245 or 246, wherein distorting the mammalian cell comprises confining the mammalian cell in a first microfluidic channel.
248. The method of any one of embodiments 245-247, wherein applying the force to the distorted mammalian cell comprises applying the force by flowing a fluid through a second microfluidic channel.
249. A method, comprising:
   confining a mammalian cell inside of a first microfluidic channel having a volume less than 120% of the cell and a maximum cross-sectional dimension less than 60% of the average diameter of a perfect sphere having the same volume as the cell; and
   flowing the confined mammalian cell through a second microfluidic channel intersecting with the first microfluidic channel.
250. A method, comprising:
   confining a mammalian cell inside of a first microfluidic channel having a volume less than 120% of the cell and a maximum cross-sectional dimension less than 60% of the average diameter of a perfect sphere having the same volume as the cell; and
   applying a force to a stretched portion of the confined mammalian cell by flowing a fluid through a second microfluidic channel.
251. A method of lysing a cell, comprising:
   confining a mammalian cell inside of a first microfluidic channel having a volume less than 120% of the cell and a maximum cross-sectional dimension less than 60% of the average diameter of a perfect sphere having the same volume as the cell; and
   applying a force to the confined mammalian cell sufficient to lyse the cell.
252. A method, comprising:
   confining a mammalian cell inside of a first microfluidic channel such that it has an aspect ratio of at least 2:1; and
   flowing the confined mammalian cell through a second microfluidic channel intersecting with the first microfluidic channel.
253. A method, comprising:
   confining a mammalian cell inside of a first microfluidic channel such that it has an aspect ratio of at least 2:1; and
   applying a force to a stretched portion of the confined mammalian cell by flowing a fluid through a second microfluidic channel.
254. A method of lysing a cell, comprising:
   confining a mammalian cell inside of a first microfluidic channel such that it has an aspect ratio of at least 2:1; and
   applying a force to the confined mammalian cell sufficient to lyse the cell.
255. A method of (e.g., selectively) lysing cells (e.g., blood cells), the method comprising:
   distorting (e.g., deforming) a cell by flowing fluid comprising the cell dispersed therein; and
   applying pressure to the cell while the cell is distorted thereby lysing the cell.
256. The method of embodiment 255, wherein the distortion is caused by one or more channels (e.g., microchannel(s)) (e.g., first channel(s)) in a device (e.g., microfluidic device).
257. The method of embodiment 255 or embodiment 256, wherein the pressure is applied by one or more channels (e.g., microchannel(s)) (e.g., second channel(s)) in a (e.g., the) device (e.g., microfluidic device).
258. The method of any one of embodiments 255-257, wherein the cell has been contacted with a surfactant (e.g., a detergent) (e.g., a saponin) prior to the distortion (e.g., such that the cell is softer) (e.g., such that the cell has one or more lowered mechanical properties).
259. The method of any one of embodiments 255-258, comprising trapping a second biological component (e.g., bacteria or virus) released from the cell by the lysing [e.g., in a channel of a device (e.g., microfluidic device)].
260. The method of any one of embodiments 69-103, 255-259, wherein the device is a microfluidic device.
261. The method of any one of embodiments 69-103, 255-259, wherein the device is a device according to any one of embodiments 1-68.
262. The method of any one of embodiments 69-103, 255-259, 260-261, wherein the one or more biological components (e.g., wherein the first biological component and/or the second biological component) is cellular.
263. The method of embodiment 262, wherein the first biological component is cells of a first type and the second biological component is cells of a second type.
264. The method of embodiment 263, wherein the first type is cancer cells (e.g., circulating tumor cells).
265. The method of embodiment 263 or embodiment 264, wherein the second type is blood cells.
266. The method of any one of embodiments 69-103, 255-259, 260-265, wherein the one or more biological components comprises (e.g., wherein the first biological component is) one or more viruses, one or more bacteria, one or more fungi, one or more prokaryotic cells, one or more eukaryotic cells (e.g., plasmodium), or a combination thereof.
267. The method of any one of embodiments 69-103, 255-259, 260-266, wherein the one or more biological components comprises (e.g., wherein the first biological component and/or the second biological component comprises) particles.
268. The method of any one of embodiments 69-103, 255-259, 260-267, wherein the one or more biological components (e.g., the first biological component) are bound to particles (e.g., are viruses bound to virus-binding beads).
269. The method of any one of embodiments 69-103, 255-259, 260-268, wherein the one or more biological components comprises (e.g., the first biological component and/or second biological component is) extracellular.
270. The method of any one of embodiments 69-103, 255-259, 260-269, wherein the first biological component is intracellular.
271. The method of any one of embodiments 69-103, 255-259, 260-269, wherein the first biological component in the fluid is trapped while flowing the fluid.

The following examples are intended to illustrate certain embodiments of the present invention, but do not exemplify the full scope of the invention.

### Example 1

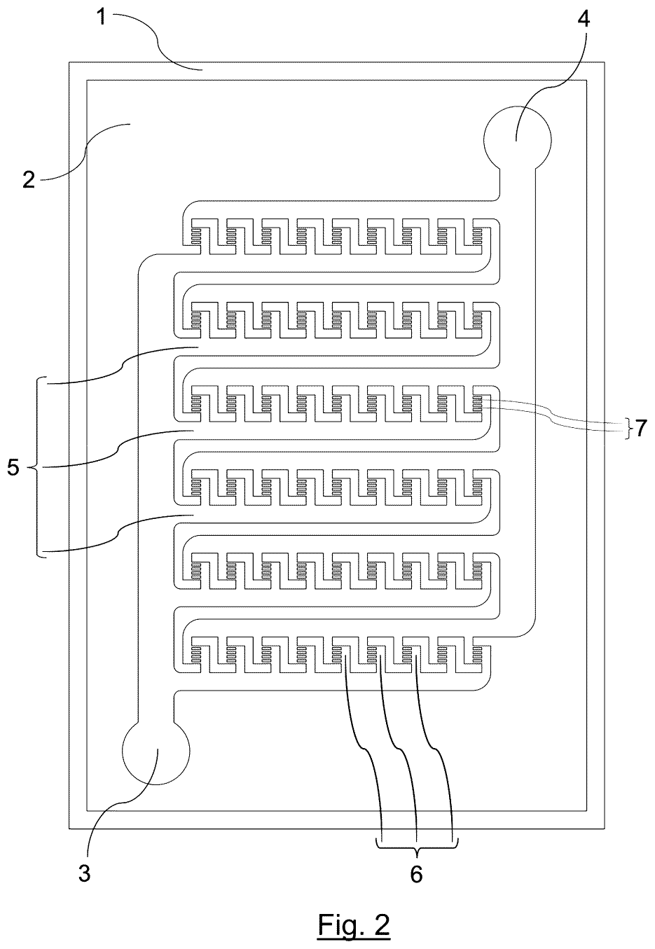

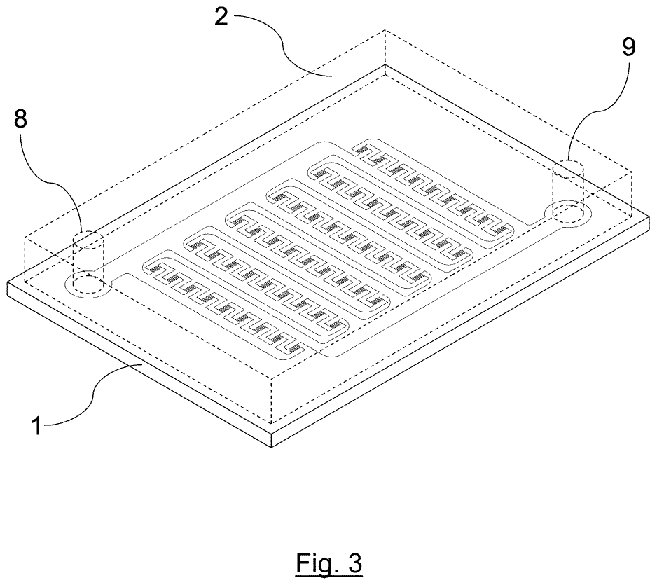

This example describes a microfluidic chip configured to separate bacteria from blood and urine. An example of a microfluidic design (FIGS. 1D, 2, and 3) includes bacteria traps 6, pinch points 7, main flow channels 5, an inlet 3, and an outlet 4. The bacteria traps are trenches large enough to accommodate bacteria of various sizes or other pathogens, typically ranging from 1 to 10 microns in width and height. The pinch points are smaller nanochannels that connect the bacteria traps to a second low-pressure region (e.g., channel). These pinch points are narrow enough (smallest dimension less than 0.4 microns) to prevent bacteria from passing through while allowing blood cells to squeeze through. Optional pretreatment of blood with a detergent can facilitate blood cell removal and passage through the filter.

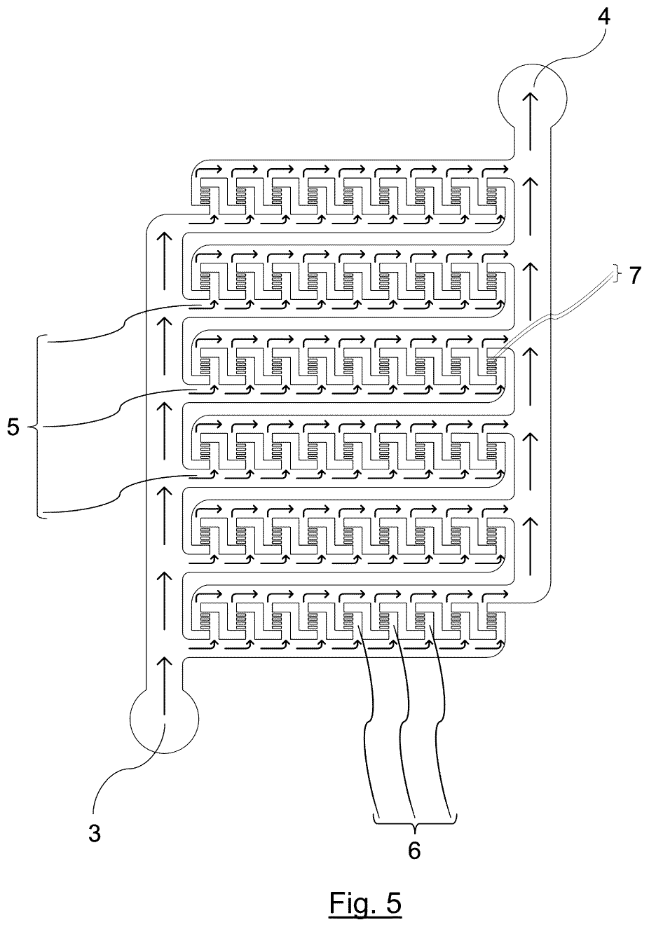

The main flow channels 5 link the bacteria traps 6 and pinch points 7 to the inlet 3 and outlet 4. In this design, the incoming flow moves from the inlet 3 into the main flow channels 5, through the bacteria traps 6 and pinch points 7, into additional main flow channels, and finally out through the outlet 4. No flow can bypass the bacteria traps 6 and pinch points 7 by going directly from inlet 3 to outlet 4 (FIG. 5). To reduce hydraulic resistance and allow high flow rates, the bacteria traps 6 and pinch points 7 are arranged in parallel, with a high density, typically from several hundred thousand to millions of bacteria traps and five to fifty times more pinch points. The main flow channels 5 are also organized in a parallel system, typically 84 or more channels, each containing more than 3,000 bacteria traps 6 and more than 15,000 pinch points 7, and are characterized by an even flow distribution across the channels.

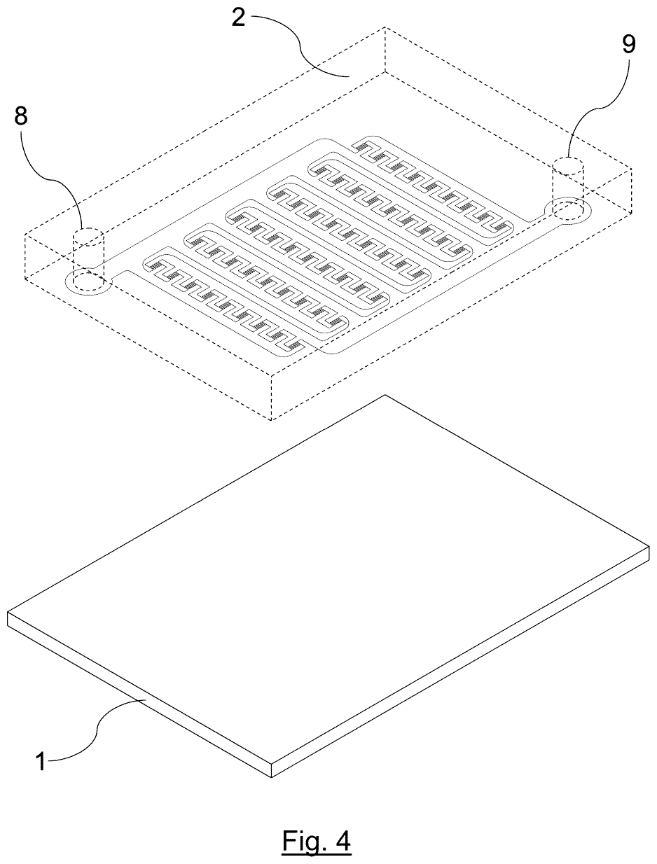

This device is fabricated by molding a suitable polymer 2 from a master mold and bonding it to a coverslip 1 with the features facing the coverslip (FIG. 4). Commonly the polymer is PDMS. The typical master mold is a silicon wafer containing etched or photoresist-based structures, ideally using SU-8 or AZ photoresists.

The microfluidic device is symmetrical, allowing the inlet to function as an outlet and vice versa. Blood (typically diluted and/or pretreated with detergent), prefiltered urine with 10-micron filter (to remove potential epidermal tissue and/or uroliths), water, and other liquids potentially containing pathogens (e.g., bacteria and/or fungi) are injected into the inlet 3. This can be done manually with a syringe or automatically using pumps, air pressure controllers, or syringe pushers. The flow rate is typically high, ranging from 1 to 5 mL/min. Due to parallelization, the flow splits and passes through numerous bacteria traps 6 and pinch points 7 (FIGS. 5 and 6). Bacteria 10 enter the traps 6 and are retained there, unable to pass through the smaller pinch points 7 (FIG. 6). Most (e.g., at least 80%, at least 90%, at least 95%, or at least 99%) or all blood cells 16 may be lysed by the pinch points and cell lysate components can pass through the pinch points and exit the device through the outlet. One reason is that blood cells 16 are much softer than bacteria 10 and can be distorted (e.g., squeezed) by the narrow pinch points 7, for example due to pressure (e.g., capillary pressure), to the point of lysing. In some embodiments, larger pinch points may allow some blood cells to pass through pinch points 7 while still trapping harder biological components, such as bacteria, as shown in FIG. 6. Small aggregates and other media components will also pass through the pinch points if they are smaller than the pinch point cross-section.

Once all liquid is filtered, the device can optionally be washed with a buffer injected through the inlet. This wash retains the bacteria 10 in the traps while clearing most residual blood cell components. The trapped bacteria 10 in 13 can be directly visualized and analyzed under a microscope. Bacterial drug susceptibility or growth rate can be directly measured by introducing different drugs or growth media through the inlet 3. The bacteria can be identified by microscopic observation or by using Fluorescence In Situ Hybridization (FISH) probes. Most or all bacteria 10 in 13 can be retrieved from the filter by reversing the flow and injecting buffer from the outlet to the inlet (FIG. 7).

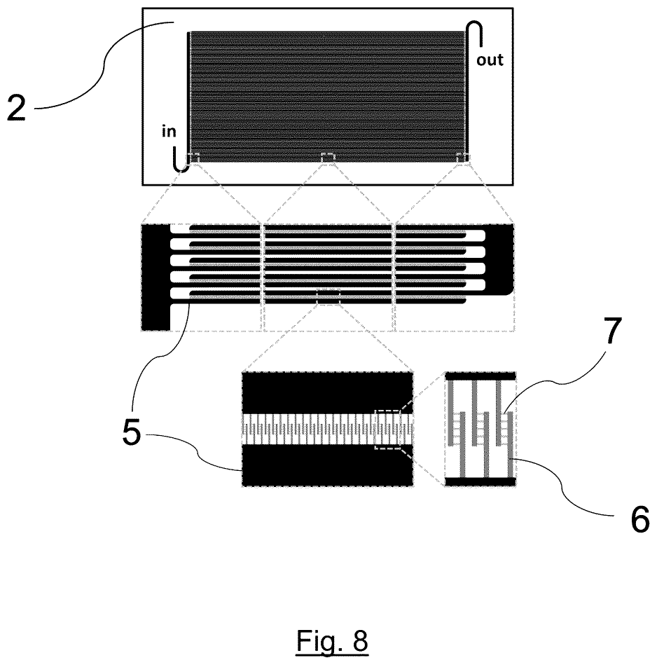

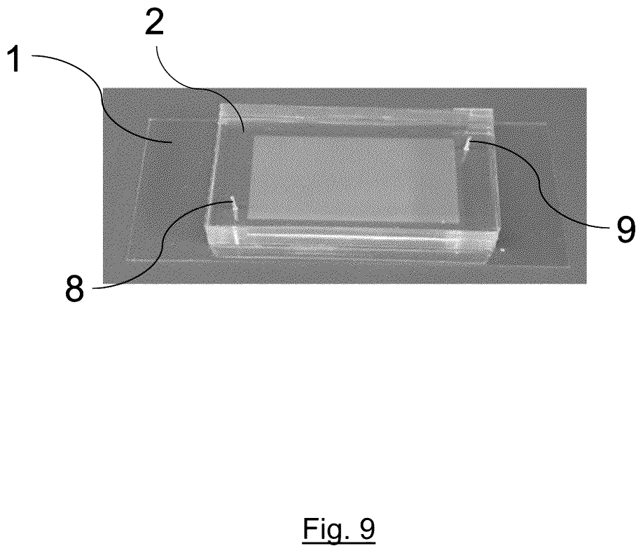

A version of the microfluidic design was fabricated for testing purposes (FIGS. 8 and 9). This version included an inlet 8, an outlet 9, 84 parallel large channels 5, each connected to 3,363 bacteria traps 6, with each trap connected to 5 pinch points 7, for a total of 282,492 bacteria traps 6 and 1,412,460 pinch points 7.

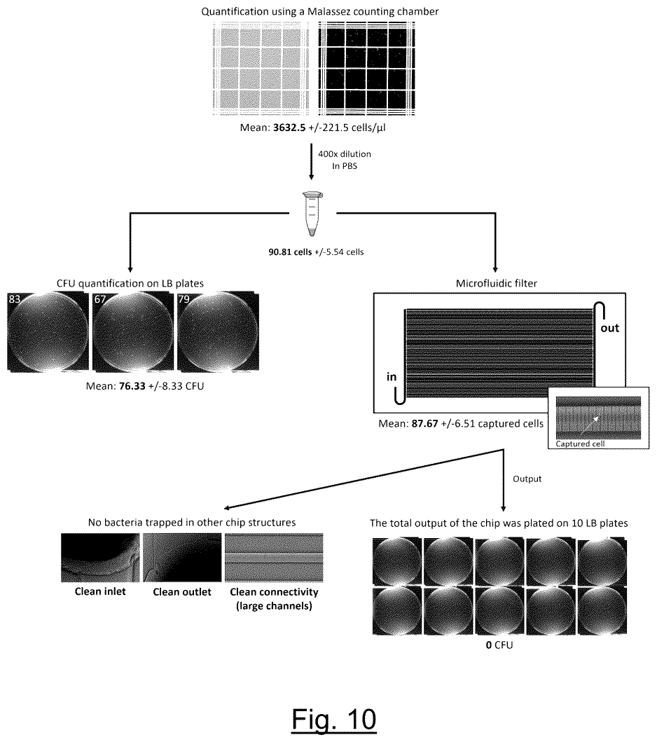

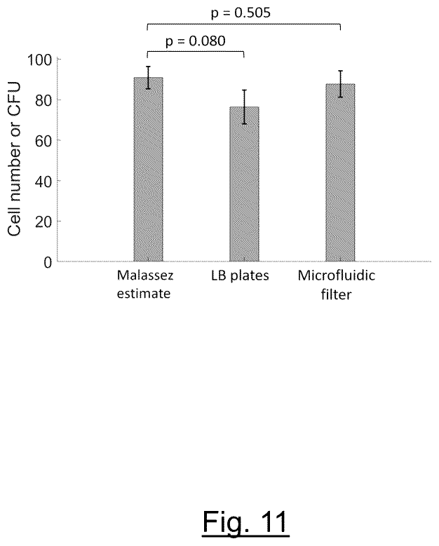

To test whether the microfluidic device can be used to isolate and detect all bacteria from a highly dilute sample, YFP-marked E. coli bacteria were grown overnight and diluted in phosphate-buffered saline (PBS). The number of bacterial cells per microliter was estimated using Malassez counting chambers (FIG. 10). After an additional 400-fold dilution, 10-microliter aliquots containing an average of 90.81±5.54 cells were prepared. Three of these aliquots were plated on LB agar plates after adding 100 microliters of PBS to each to facilitate plating. Three other aliquots were injected with a rate of 5 mL/min into three different casts of the microfluidic device after adding 3 mL of PBS to each (estimated final bacterial density of 30.17 cells/mL). The mean number of colony-forming units (CFUs) on the plates was compared with the mean number of bacteria trapped and detected in the microfluidic device and with the estimated number of bacteria in the aliquots (FIG. 11). All three numbers were not statistically different. An average of 87.67±6.51 cells were trapped in the microfluidic filter, while 76.33±8.33 CFUs were observed on the plates.

As a control, the 3 mL output of the filter was plated, and no CFUs were detected on the plates, showing that bacteria do not escape from the filter. Moreover, the inlet, outlet, and large channels were carefully checked under the microscope for any remaining bacteria, and none were found.

These results suggest that all bacteria injected into the filter are trapped and detected and confirm that the filter can be used with a high flow rate. This also suggests that the detection limit of the microfluidic device is lower than 5×10⁻²⁰ M, which is desired for diagnostic tools.

To further assess the trapping efficiency of the filter, a filter containing exactly 88 cells was unloaded by reversing the flow and passing 5 mL of PBS from the outlet to the inlet (FIG. 12). This flow was directly injected into another empty filter. After all the flow was passed through the second filter, the bacteria were counted in both filters. Zero bacteria were found in the first chip that originally contained 88 cells, suggesting that the chip was successfully fully unloaded. All 88 cells were found in the second filter, demonstrating 100% trapping efficiency.

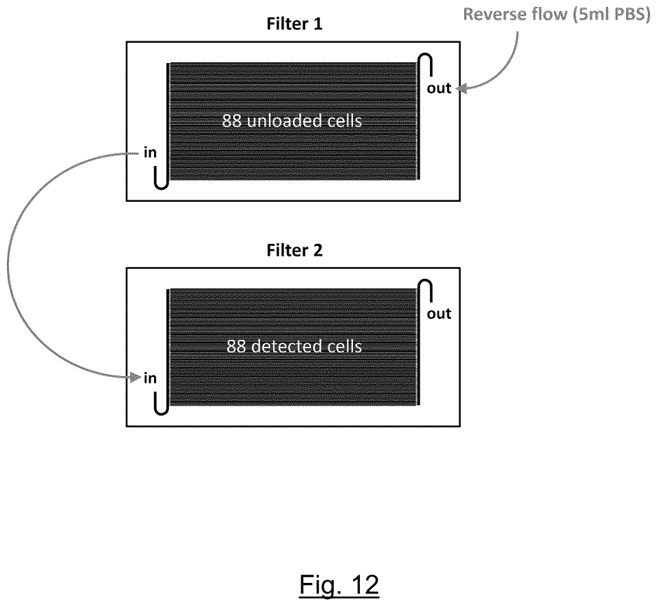

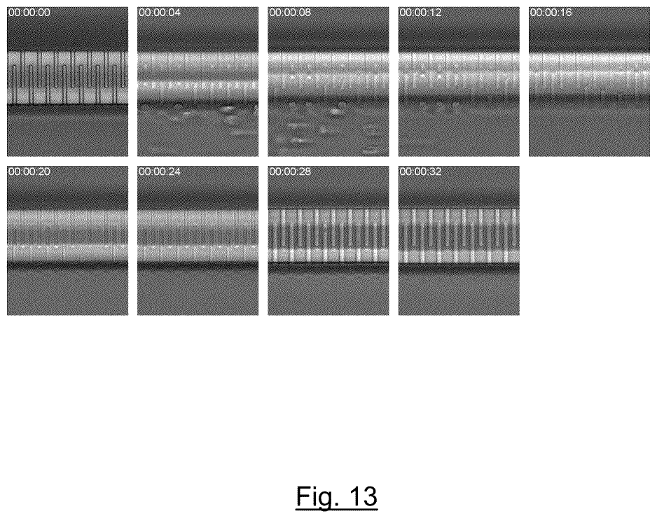

The microfluidic device was tested with negative human blood (FIG. 13). 0.4 mL of blood were diluted in 1.2 mL PBS (1.6 mL final volume) and injected into the filter, followed by 1.5 mL pure PBS to wash the filter. A real-time movie was acquired. This movie confirmed that the blood cells were able to pass through the filter pinch points. After PBS washing, the bacteria traps appeared relatively clean of cell debris.

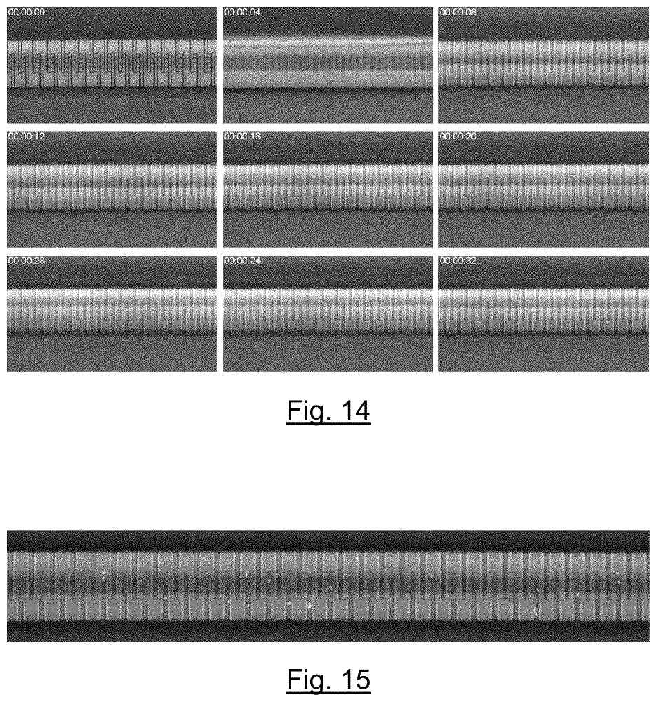

Next, the filter was tested with blood spiked with YFP-marked E. coli (FIGS. 14 and 15). To do this, 0.375 mL of human blood was mixed with 0.005 mL of ten-fold diluted overnight YFP-marked E. coli culture, 0.075 mL of 20% saponin solution (0.5% final concentration), and 2.545 mL PBS. The mixture was injected at a flow rate of 5 mL/min into the microfluidic device. No blood cells were detected in the bacteria traps after passing the 3 mL mixture through the filter. However, E. coli cells were detected in the bacteria traps, while no CFUs were detected in the output, suggesting that all bacteria were retained in the filter.

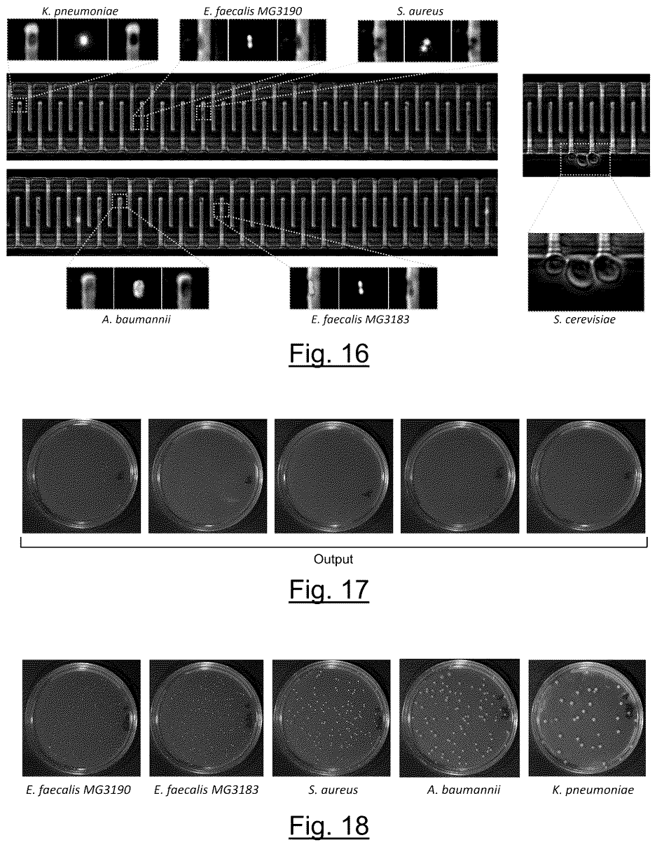

To test whether the chip can retain different bacterial strains or fungi, human blood was spiked with five different pathogenic bacterial strains and budding yeast: E. faecalis MG3190, K. pneumoniae, A. baumannii, E. faecalis MG3183, S. aureus, and S. cerevisiae (FIG. 16). The bacteria were spiked into blood and then mixed together. To differentiate them, different fluorescent reporters or dyes were used: mCherry (two strains), CFP, GFP, and Cy5 (dye). The two strains marked by mCherry were easily distinguishable by their shape and size. Before mixing the bacteria, the same volume of spiked blood with each strain was plated to quantify the CFUs. After saponin was used to lyse the blood cells, the mixture was injected into the microfluidic filter. The output of the chip was plated and showed no CFUs (FIG. 17). The 5 different bacteria strains were all detected in the filter and counted. For each strain, the number of bacteria detected in the filter was compared with the number of CFUs contained in the spiked blood before mixing and injecting into the filter (FIG. 18). The results indicate that approximately 28% more bacteria are found in the filter than on the plates, confirming the exceptional retention rate of the filter.

Similarly, budding yeast was spiked into blood, treated with saponin (0.5%), and injected into the microfluidic filter (FIG. 16). Yeast cells were successfully detected in the filter, while no CFUs were observed in the output plated on YPD-agar. This suggests that the filter is effective at isolating fungi.

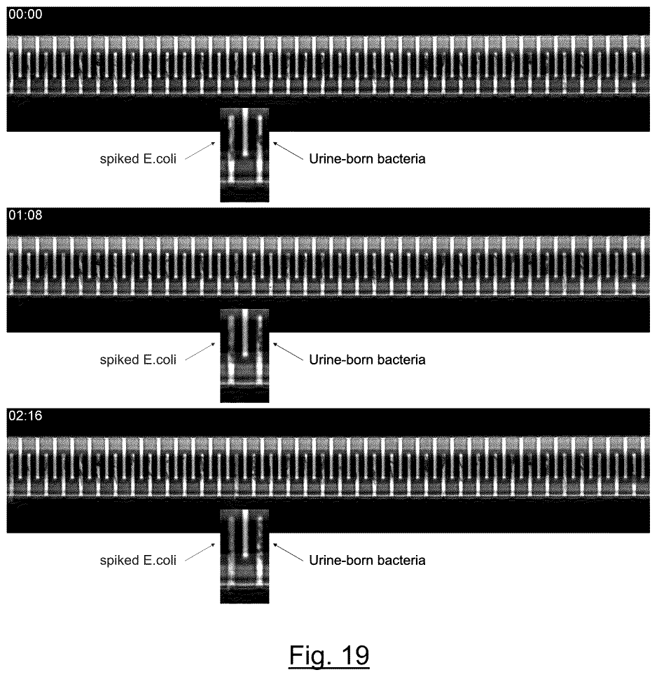

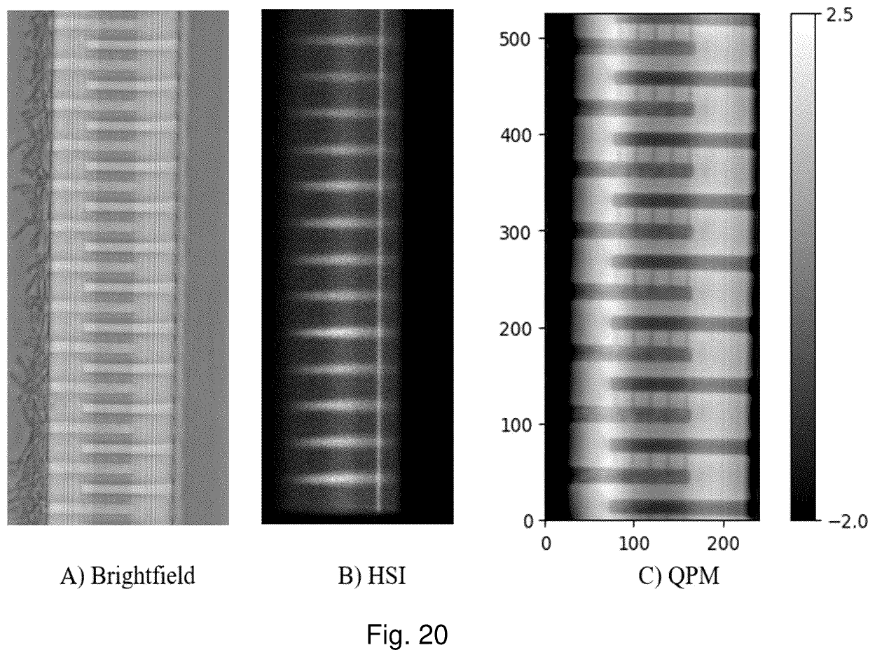

To test whether the filter could isolate bacteria from a urine sample, mCherry-marked E. coli was spiked into human urine (FIG. 19). A 2 ml portion of this solution was prefiltered with a 5-micron filter to remove epidermal cells and tissue debris, and then injected into the filter. Both mCherry-positive and mCherry-negative bacteria were detected within the filter, with the mCherry-negative bacteria corresponding to urine-borne bacteria.

The output from the chip was plated, and no CFUs (colony-forming units) were detected, suggesting that all spiked or urine-borne bacteria were retained by the filter. Growth media was subsequently flowed through the chip, and bacterial growth was confirmed. This indicates that the filter can be directly used to extract bacterial growth rates.

### Example 2

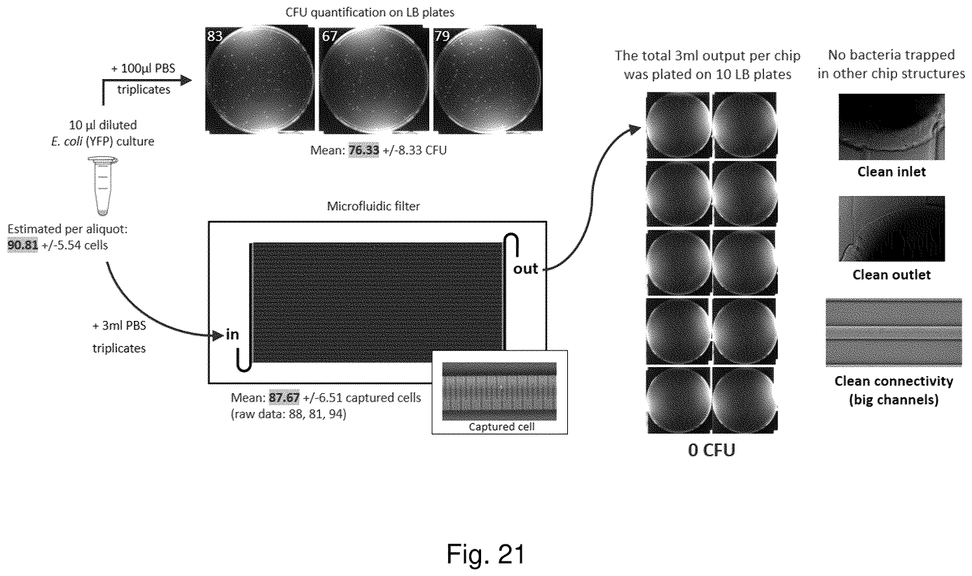

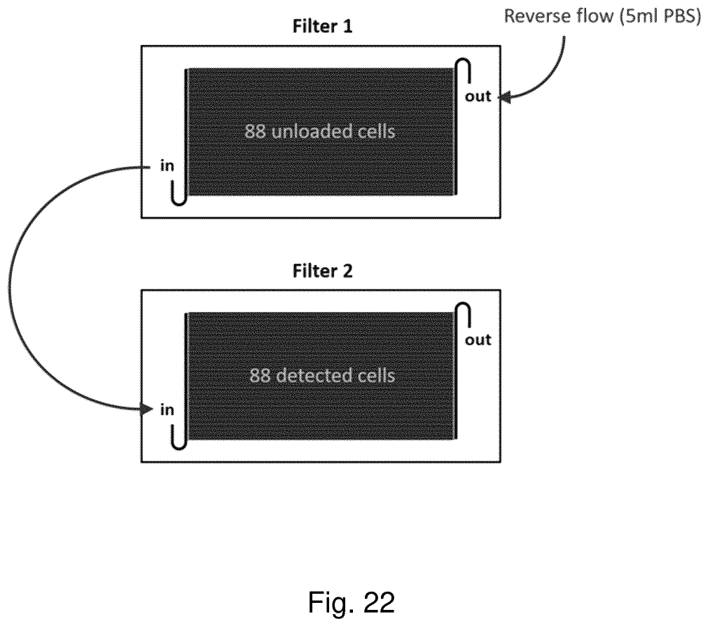

This example describes microfluidic devices, such as microfluidic filters, for trapping bacteria. Some devices are optically clear sub-micron filters, where fluid goes through ‘backports’ or ‘sideports’ that can be just a few hundred nanometers in size. Even the smallest bacteria are too large to go through, much like standard 0.2 μm filters. We confirmed that experimentally by multiple methods (FIG. 21). First, we flowed high concentrations of bacteria and plated all fluid that went through, to confirm that not a single bacteria passed the filters. Second, we imaged the entire chip with sub-micron resolution to confirm that no bacteria could hide in inlets and outlets. Third, we performed conventional tests of running many test samples, plating cells directly on agar plates for CFUs/ml and compared those counts with counts from the microfluidic chips. This yielded virtually identical numbers, down to the 5-10% Poisson counting errors given the abundances used. Finally, we subjected the chips to a more rigorous test. Specifically, we filtered a sample and identified exactly 88 bacterial cells trapped. We then retrieved all these cells from the chip, and added them to a second sample that was then filtered through a second device. In the second device we retrieved every cell (88 out of 88), showing 100% trapping efficiency.

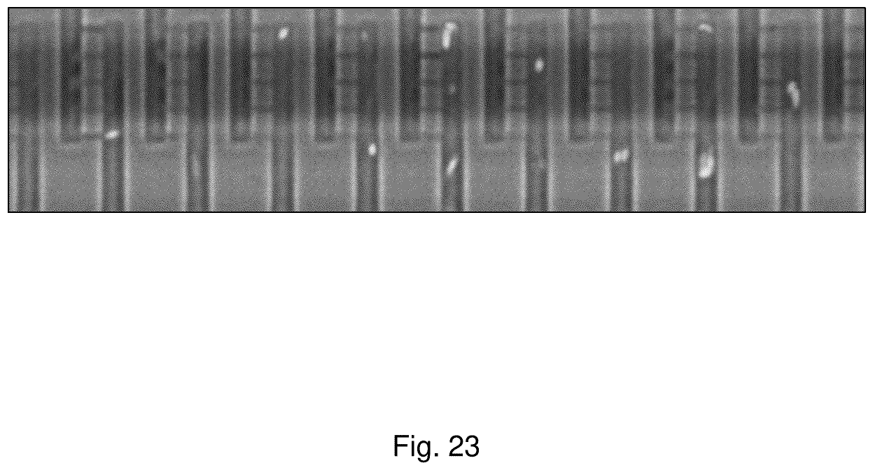

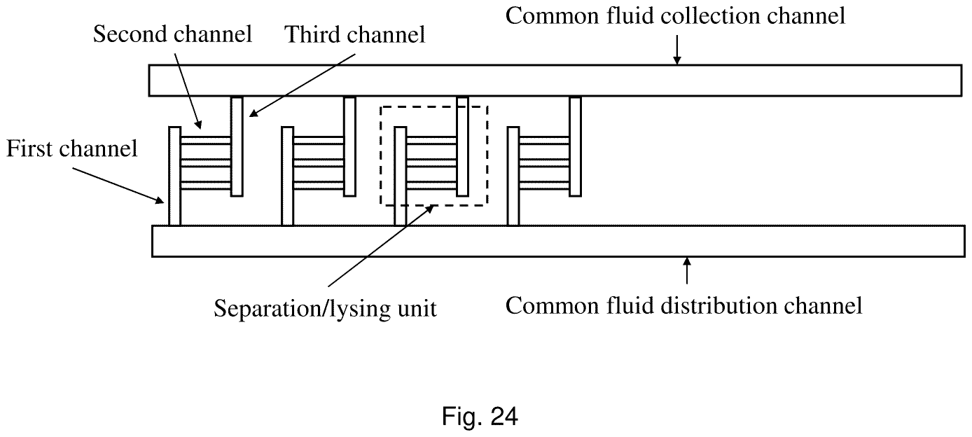

We then built filters that include 0.2 μm sieve holes but further include several other features that modify the shape of blood cells to optimize their flexibility just before they enter the final 0.2 μm barriers (FIG. 23). These features, which in some ways resemble the architecture of spleens strikingly allowed us to eliminate not just red blood cells, but also white blood cells—and any other human part or debris from our fresh, whole human blood spiked with microbes.

This performance is made possible by combining multiple features. We include systems on three distinctly different length-scales, and the exact combinations of shapes and dimensions matter at all scales. We also include a very large number of filter holes—over 3 million in the first generation of chips built, each made to specifications down to around 10 nm resolution with precise shapes. The flow systems were computationally optimized to reduce resistance to maximize sample throughput. In some instantiations, the blood was diluted with PBS or small amounts of detergents were added, but much less and for shorter times than in our other tests, with no effect on the microbes tested.

These filters show exceedingly efficient and precise elimination of every single human cell in fresh whole blood—even for samples containing close to 10¹¹ such cells—while also trapping every bacterial cell. Second, they require no instruments—not even electricity, but simply the user to inject blood with a syringe into a consumable. Third, that consumable could be mass-produced for a few dollars or less. Fourth, these chips are optimized for imaging and traps the microbes in a small part of the chip, that can be optically inspected in minutes or less, to count the exact number of microbes, regardless of species, and thereby shows immediately if a sample is negative. This in particular provides clinicians with robust evidence to avoid unnecessarily admitting patients to hospital. Every foreign cell in the blood is individually accounted for. Fifth, the flow rates are amazingly high, and we already handle 1-2 ml/min, but should be able to increase that almost 10-fold or 100-fold by simple parallelization and other optimizations.

### Example 3

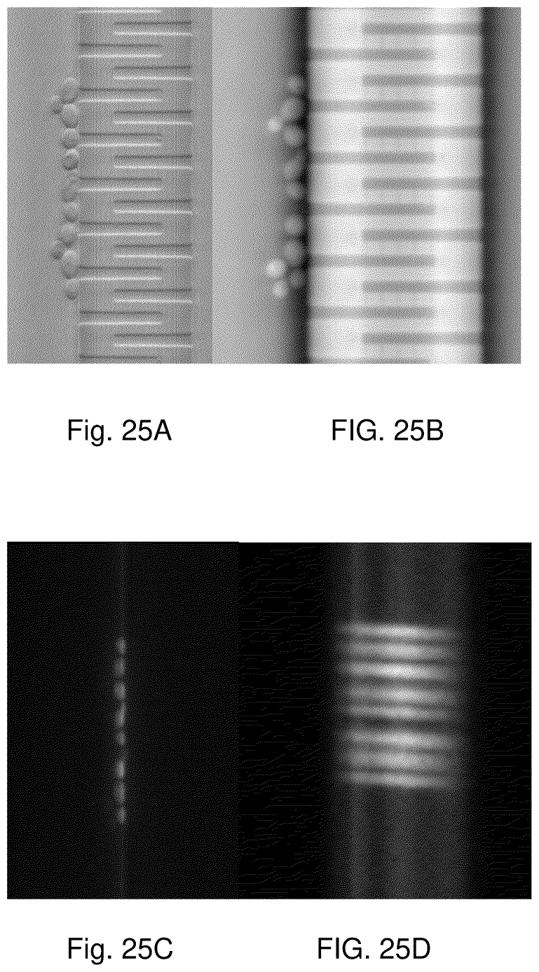

In this example, Candida albicans was isolated using a device in accordance with one embodiment. FIG. 25 shows that Candida albicans has been physically isolated within a filter-type chip architecture and sequentially imaged using (FIG. 25A) brightfield, (FIG. 25B) quantitative phase microscopy (QPM), (FIG. 25C) image of cell excitation with the hyperspectral laser, and (FIG. 25D) hyperspectral imaging (HSI) emission, according to illustrative embodiments of the present disclosure. Images were captured in the same field of view sequentially (FOV) on an integrated imaging system. Cells were observed to shift slightly during imaging due to fluid flow through the device which confirms the ability to easily remove trapped cells by reversing the flow.

### Example 4

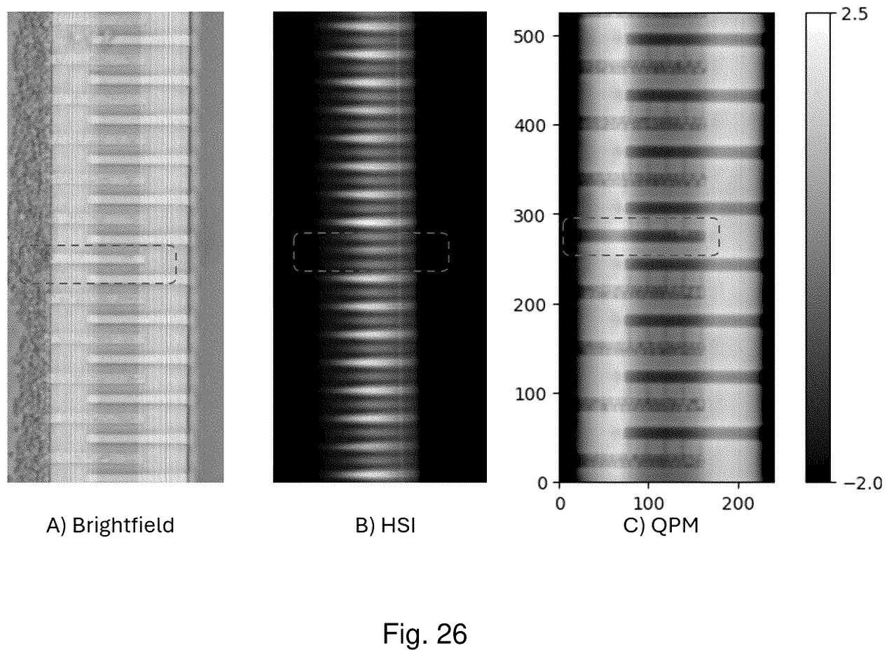

FIG. 26 illustrates a gram-positive Staphylococcus aureus that has been physically isolated within trench geometries in a device comprising first and second channels and sequentially imaged using (A) brightfield, (B) hyperspectral imaging (HSI), and (C) quantitative phase microscopy (QPM). Images were captured in the same field of view sequentially (FOV) on an integrated imaging system. Noted in the outlined box is a trench with a decreased HSI response which aligns with a decreased number of S. aureus cells visualized in both brightfield and QPM. As observed, and as seen in panel (A), once the trenches had been filled with bacteria, fluid still flowed readily through the channels of the device as more bacteria cells accumulate on a surface of a common fluid distribution channel (e.g., at entrances to first channels) visible on the left of panel (A). Reverse flow through the device could be used to retrieve a substantial majority or all of the bacteria.

### Example 5

FIGS. 28A-B illustrate quantitative phase microscopy images of Escherichia coli bacteria trapped within trench geometries in a microfluidic device comprising multiple microfluidic filters each comprising microfluidic channels comprising a first channel, second channels, and a third channel. A fluid comprising the bacteria in growth media seeded at 100,000 CFU/mL was flowed through the microfluidic channels. The bacteria were exposed to a flow pressure of 30 mbar from an inlet (inlet not shown) to a common fluid distribution channel 2810 then into a first channel 2820 then into second channels 2830a-d. Fluid flowed from vertically oriented second channels 2830a-c into a third channel 2840 then into a common fluid collection channel 2850. Fluid flowed from horizontally oriented second channel 2830d directly into the common fluid collection channel 2850. (For clarity, in FIGS. 28A-B a single label is used to collectively label second channels 2830a-c). The first channels have a width and height of 3 μm by 1.8 μm, respectively. The second channels have a width and height of 2 μm by 0.5 μm, respectively. As shown in FIGS. 28A-B, bacteria injected into the filter were successfully trapped in the first channels, before the second channels, in each microfluidic filter, demonstrating the trapping efficiency of the microfluidic filters. The bacteria were subsequently imaged using quantitative phase imaging (QPM), demonstrating the microfluidic device's capability to operate with singular and/or multimodal microscopy imaging and the ability to detect bacteria trapped in the microfluidic filters. Thes capabilities may each allow identification, characterization, and/or tracking of bacteria cells (e.g., within a microfluidic device).

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When the word “about” is used herein in reference to a number, it should be understood that still another embodiment of the disclosure includes that number not modified by the presence of the word “about.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1-8. (canceled)

9. A device for separating and/or lysing one or more biological components in a fluid, the device comprising first channels and second channels arranged such that fluid flow through the device travels through the first channels then the second channels, wherein the first channels are larger than the second channels.

10. The device of claim 9, wherein each of the first channels have at least one dimension that is larger than a corresponding at least one dimension of each of the second channels.

11. (canceled)

12. The device of claim 9, wherein a ratio of at least one dimension in the first channel to the corresponding at least one dimension in second channel is in a range of from 1 to 1000.

13. (canceled)

14. The device of 9, wherein the second channels are arranged at an angle relative to the first channels in a range of from 0 to 180 degrees.

15. The device of claim 9, wherein each of the first channels is directly fluidically coupled to at least one of the second channels.

16. The device of claim 15, wherein the at least one of the second channels is a plurality of the second channels, wherein the plurality of the second channels are distributed over a distance of no more than 100 μm.

17-20. canceled

21. The device of claim 15, wherein, for each of the first channels, the at least one of the second channels are disposed relative to the first channel to apply a pressure gradient to a side of a biological component when disposed in the first channel.

22. (canceled)

23. The device of claim 9, wherein the first channels have a width and/or a height in a range of from 50 nm to 15 μm.

24. (canceled)

25. The device of claim 9, wherein the second channels have a width and/or a height in a range of from 20 nm to 800 nm.

26. (canceled)

27. The device of claim 9, comprising third microchannels larger than the second channels, wherein fluid flow through the device travels through the second channels then the third microchannels.

28-29. (canceled)

30. The device of claim 27, wherein the third microchannels are directly fluidically coupled to a common fluid collection channel.

31-32. (canceled)

33. The device of claim 9, comprising a common fluid distribution channel network, wherein each of the first and each of the third microchannels are directly fluidically coupled to the common fluid distribution channel network.

34. (canceled)

35. The device of claim 9, wherein the first channels and the second channels are sized and arranged such that at least 25% bacteria in fluid that is flowed through the first channels and the second channels are separated.

36-38. (canceled)

39. The device of claim 9, comprising biological component separation units comprising ones of the first channels and ones of the second channels and/or biological component lysing units comprising ones of the first channels and ones of the second channels.

40. (canceled)

41. The device of claim 39, wherein the units comprise channels arranged such that fluid flow through the units causes selective lysing of one or more biological components.

42. The device of claim 39, wherein the plurality of units comprises at least 1,000 connected in parallel.

43-45. (canceled)

46. The device of claim 9, comprising the fluid disposed in the first channels and the second channels, wherein the fluid comprises a lysing composition.

47-48. (canceled)

49. The device of claim 9, wherein the first channels and the second channels are arranged to trap one or more infectious agents or target cells in and/or at the first channels.

50. The device of claim 9, wherein the first channels and the second channels are arranged to lyse one or more cellular biological components using the first channels and the second channels.

51-271. (canceled)

272. The device of claim 39, comprising the biological component separation units, wherein the ones of the first channels and the ones of the second channels comprised in the units are arranged such that fluid flow through the units causes separation of biological components having different sizes and/or mechanical properties.

273. The device of claim 39, comprising the biological component lysing units, wherein the ones of the first channels and the ones of the second channels comprised in the units are arranged such that fluid flow through the units causes separation of biological components and lysing of one or more biological components.

274. The device of claim 46, wherein the lysing composition comprises a lysing agent for lysing the one or more biological components in the device.

275. The device of claim 46, wherein the lysing composition is formulated to increase separation and/or lysing of the one or more biological components in the device.

276. The device of claim 46, wherein presence of the lysing composition increases a percentage of the one or more biological components that is separated and/or lysed in the device during flow through the first channels and the second channels.

277. The device of claim 9, wherein the first channels and the second channels are arranged to reversibly trap one or more biological components.

278. The device of claim 9, wherein the first channels and the second channels are arranged to reversibly trap one or more biological components at and/or in the first channels such that the one or more biological components are removable under reverse fluid flow through the device.

279. The device of claim 9, wherein the first channels can have a size corresponding to one or more viruses, one or more bacteria, one or more fungi, one or more prokaryotic cells, one or more eukaryotic cells, or a combination thereof.

280. The device of claim 9, wherein the first channels and the second channels are arranged to trap one or more viruses, one or more bacteria, one or more fungi, one or more prokaryotic cells, one or more eukaryotic cells, or a combination thereof in and/or at the first channels.
